(19) United States
(12) Patent Application Publication
(10) Pub. No.: US 2004/0076999 A1
(43) Pub. Date: Apr. 22, 2004

(54) Computer user interface facilitating acquiring and analyzing of biological specimen traits

(75) Inventors: Edward Faeldt, Backefors (SE); Luis Serrano, Heidelberg (DE); Cayetano Gonzalez, Madrid (ES); Christian Boulin, Wiesloch (DE); Christopher J. Cummings, Brookline, MA (US); Juan Botas, Houston, TX (US); Huda Zoghbi, Houston, TX (US)

Correspondence Address: PALMER & DODGE, LLP, KATHLEEN M. WILLIAMS, 111 HUNTINGTON AVENUE, BOSTON, MA, 02199, US

(73) Assignees: Baylor College of Medicine; European Molecular Biology Laboratory (EMBL); EnVivo Pharmaceuticals

(21) Appl. No.: 10/619,227

(22) Filed: Jul. 14, 2003

Related U.S. Application Data
(60) Provisional application No. 60/396,064, filed on Jul. 15, 2002.
(60) Provisional application No. 60/396,339, filed on Jul. 15, 2002.

(51) Int. Cl. C12Q 1/68; G06F 19/00; G01N 33/48; G01N 33/50

(52) U.S. Cl. 435/6; 702/20

(57) ABSTRACT

Tools are provided for obtaining and assessing data concerning the physical or behavioral traits of an biological specimen population for the purpose of identifying, treating, or gathering intelligence on the condition of the specimen population. In one aspect, a computer system is provided to assess a condition of a biological specimen by studying the physical traits of a sample that comprises a number of specimens. The condition may comprise a human central nervous system condition. A user interface is provided that comprises a computer screen, an input interface portion, and a processing mechanism. The user interface may further comprise a specimen information input mechanism. The specimen information input mechanism may comprise a specimen type input that allows the user to specify, through the computer screen input, the type of specimen to be studied.

### RELATED APPLICATION DATA

This application claims priority to U.S. Provisional Applications: No. 60/396,064 filed on Jul. 15, 2002, and 60/396,339 filed on Jul. 15, 2002. The content of each of these applications is hereby expressly incorporated by reference herein in its entirety.

### BACKGROUND

1. Copyright Notice.

This patent document contains information subject to copyright protection. The copyright owner has no objection to the facsimile reproduction by anyone of the patent document or the patent, as it appears in the U.S. Patent & Trademark Office files or records but otherwise reserves all copyright rights whatsoever.

2. Field of the Invention

Aspects of the invention relate to tools for gathering data regarding the visible features of biological species. Other aspects relate to tools for assessing an animal's condition or for assessing a treatment and its effect on an animal's condition.

3. Discussion of Background Information

There are biological assaying processes, used, e.g., in drug screening and drug discovery, that involve the use of imaging technologies. At one level, machine vision is used to identify visible features of animals (e.g., behavior, by tracking motion). At a more minute level, cell imaging techniques are used, employing a light microscope, to identify visible features of cells.

By way of example, there are a number of existing systems that use imaging to monitor the behavior of an animal, to facilitate the study of central nervous system conditions. The Dynamic Image Analysis System (DIAS) is a system for dynamic analysis of moving objects, and calculates parameters about the shape and motion of the object using the contour and path of the object. DIAS analyzes the dynamic changes in an object (U.S. Pat. No. 5,655,028). EthoVision, produced by Noldus Information Technology, Inc., is an automated video tracking system used in animal behavior experimentation for quantifying motion, including speed, distance moved, and turning of an animal. The animal is tracked on the basis of color or contrast with a reference image of the background, and the maximum number of animals that can be tracked is sixteen (www.noldus.com/products/ethovision/ethovision.html; updated Jan. 28, 2002).

### SUMMARY

The present invention is directed to tools for obtaining and assessing data concerning the physical or behavioral traits of an biological specimen population for the purpose of identifying, treating, or gathering intelligence on the condition of the specimen population (e.g., a central nervous system or neurodegenerative condition).

In one aspect of the invention, a computer system is provided to assess a condition of an animal specimen (or cell, or another biological specimen) by studying the physical traits of a sample that comprises a number of specimens. The condition may comprise a human central nervous system condition. As an example, the sample may comprise a number of transgenic non-human animal specimens. A user interface is provided that comprises a computer screen, an input interface portion, and a processing mechanism. The user interface may further comprise a specimen information input mechanism. The specimen information input mechanism may comprise a specimen type input that allows the user to specify, through the computer screen input, the type of specimen to be studied.

The specimen information input mechanism may comprise a sample identification input, e.g., comprising a manual input through the computer screen, or an automatic assignment mechanism. Additionally, or in the alternative, a bar code input may be used. The user interface may further comprise a physical trait input mechanism that allows the user to specify, through the computer screen input, a set of physical traits of the sample to be determined.

A motion tracking system may be provided to monitor the movements and behavior of the biological specimens by tracking motion of the specimens within the sample and producing motion information. From the motion information, the motion tracking system produces (e.g., stores or displays) motion-related physical trait data concerning the set of physical traits input through the physical trait input mechanism. The data storage comprises sample identification data, and the produced physical trait data corresponding to the sample.

### BRIEF DESCRIPTION OF THE DRAWINGS

FIG. 1 is a flowchart of a test and reference animal population comparison process.

FIG. 2 is a system diagram of an embodiment of an animal trait assaying system.

FIG. 3 is a system diagram of an embodiment of an assaying computer system.

FIG. 4 is a flow diagram of a user interface process.

FIG. 5 is a schematic screenshot of an embodiment of a user interface for inputting assaying parameters.

FIG. 6 is a schematic screenshot of the trait type input mechanism.

FIG. 7 is a flow diagram of an exemplary process for processing and analyzing a digitized movie.

FIG. 8 is a flow diagram of a process for processing a frame.

FIG. 9A is an exemplary frame of a digitized movie.

FIG. 9B is an exemplary background approximation of an exemplary frame of a digitized movie.

FIG. 9C is an exemplary binary image of an exemplary frame of a digitized movie.

FIG. 9D is a normalized sum of a set of exemplary binary images.

FIG. 10 is an exemplary image block.

FIG. 11 is a flow diagram of an exemplary process for tracking the motion of an animal population.

FIG. 12 is an exemplary trajectory.

FIGS. 13A-13B show assigning an exemplary trajectory to an exemplary image block.

FIG. 14 shows assigning two exemplary trajectories to an exemplary image block.

FIGS. 15A-15E are exemplary frames of a digitized movie.

FIGS. 16A-16E are exemplary graphic representations of image blocks deduced from binary images of the exemplary frames depicted in FIGS. 16A to 16E.

FIGS. 17A-17D are exemplary graphic representations of image blocks.

FIG. 18 shows exemplary trajectories.

FIG. 19 is an exemplary amount of turning.

FIGS. 20A-20B show an exemplary amount of stumbling.

FIG. 21 is a schematic representation of an exemplary data structure for the assay data.

FIGS. 22A-22B show illustrative start view screen shots.

FIG. 23 shows an exemplary grouping view screen shot.

FIG. 24 is a group setting dialog box.

FIG. 25 is a general setting dialog box.

FIGS. 26A-26C show illustrative board view screen shots.

FIGS. 27A-27C show illustrative bars view screen shots.

FIGS. 28A-28C show illustrative group view screen shots.

FIGS. 29A-29C show illustrative trial view screen shots.

FIGS. 30A-30B show illustrative sample view screen shots.

FIG. 31 is an automation control screen shot.

FIG. 32 is a bar graph from Example 2 showing the results of an assay of treated and control flies.

FIG. 33 is a line graph from Example 3 showing motor performance, assessed by the Cross150 score (y-axis) plotted against time (x-axis).

FIGS. 34A-34J from Example 3 are ten plots showing the average p-values for different populations for each combination of a certain number of video repeats and replica vials.

FIG. 35 from Example 4 is a line graph showing motor performance on the y-axis (Cross150) plotted against time on the x-axis (Trials).

### DETAILED DESCRIPTION

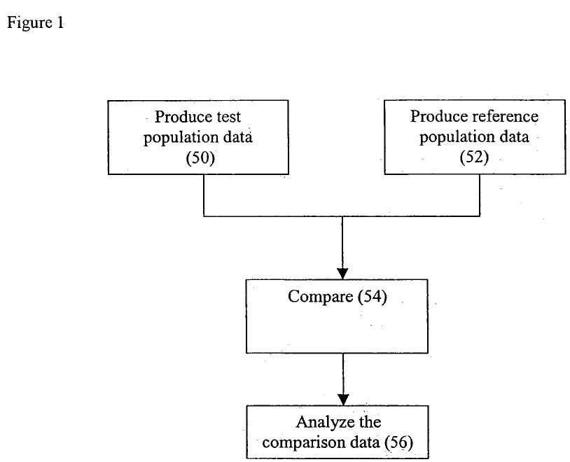

Referring now to the drawings in greater detail, FIG. 1 shows an biological specimen population comparison process for assessing a condition or treatment of a condition, involving a test population and a reference population. In acts 50 and 52, test population data and reference population data are obtained, respectively.

In one embodiment, the test population comprises an animal population with a central nervous system condition, and the reference population does not have the condition. More specifically, e.g., the test population gene predisposing it to a central nervous system condition, and the reference does not have this gene. Both populations are given a treatment before the data set is obtained.

In another embodiment, the test population is given a treatment for a central nervous system condition and the reference is not given the treatment.

In act 54, the data sets from the test and reference populations are compared, and the comparison is analyzed in act 56.

In one embodiment, the analysis in act 56 uses a threshold value to determine if there is a difference between the test and reference populations. For example, if the test population has a central nervous system condition and the reference does not, then if the differential of motion traits between the two populations is above a specified threshold, those motion traits can be considered to indicate the presence of the central nervous system condition afflicting the test population.

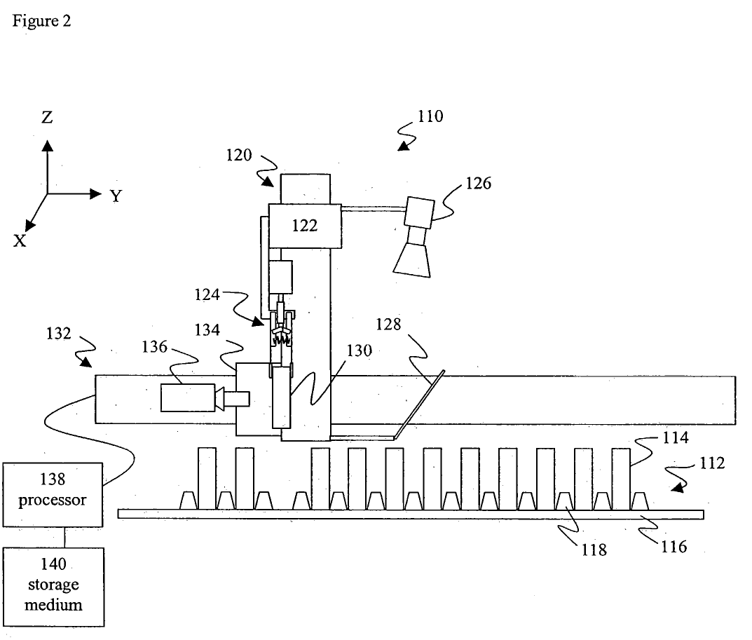

FIG. 2 shows an exemplary animal trait assaying system 110. As described below in greater detail, assaying system 110 can operate to monitor the activity of samples in a sample container 114. The samples held in sample containers 114 are a biological specimen population, where in this embodiment, each specimen in the sample is the same type of specimen. Further, in this embodiment the specimen population is preferably an animal population, more preferably flies, and even more preferably Drosophila. It should be noted, however, that motion tracking apparatus 110 can be used in connection with monitoring the activities of various organisms within various types of sample containers.

In one exemplary embodiment of assaying system 110, a robot 124 removes a sample container 114 from a sample platform 112, which holds a plurality of sample containers 114. Robot 124 positions sample container 114 in front of camera 136. Sample container 114 is illuminated by a lamp 126 and a light screen 128. Camera 136 then either captures a movie, or a series of images, of the activity of the specimen population within sample container 114. After the movie has been obtained, robot 124 places sample container 114 back onto sample platform 112. Robot 124 can then remove another sample container 114 from sample platform 112. A processor 138 can be configured to coordinate and operate sample platform 112, robot 124, and camera 136. As described below, system 110 can be configured to receive, store, process, and analyze the movies captured by camera 136.

In the present embodiment, sample platform 112 includes a base plate 116 into which a plurality of support posts 118 is implanted. In one exemplary configuration, sample platform 112 includes a total of 416 support posts 118 configured to form a 25×15 array to hold a total of 375 sample containers 114. As depicted in FIG. 2, support posts 118 can be tapered to facilitate the placement and removal of sample containers 114. It should be noted that sample platform 112 can be configured to hold any number of sample containers 114 in any number of configurations.

System 110 also includes a support beam 1120 having a base plate 122 that can translate along support beam 120, and a support beam 132 having a base plate 134 that can translate along support beam 132. In FIG. 2, support beam 120 and support beam 132 are depicted extending along the Z axis and Y axis, respectively. As such, base plate 122 and base plate 134 can translate along the Z axis and Y axis, respectively. It should be noted, however, that the labeling of X, Y, and Z axes in FIG. 2 is arbitrary, and provided for the sake of convenience and clarity.

In the present embodiment, robot 124 and lamp 126 are attached to base plate 122, and camera 136 is attached to base plate 134. As such, robot 124 and lamp 126 can be translated along the Z axis, and camera 136 can be translated along the Y axis. Additionally, support beam 120 is attached to base plate 134, and can thus translate along the Y axis. Support beam 132 can also be configured to translate along the X axis. For example, support beam 132 can translate on two linear tracks, one on each end of support beam 132, along the X axis. As such, robot 124 can be moved in the X, Y, and Z directions. Additionally, robot 124 and camera 136 can be moved to various X and Y positions over sample platform 112. Alternatively, sample platform 112 can be configured to translate in the X and/or Y directions.

Assaying system 110 can be placed within a suitable environment to reduce the effect of external light conditions. For example, system 110 can be placed within a dark container. Additionally, system 110 can be placed within a temperature and/or humidity controlled environment.

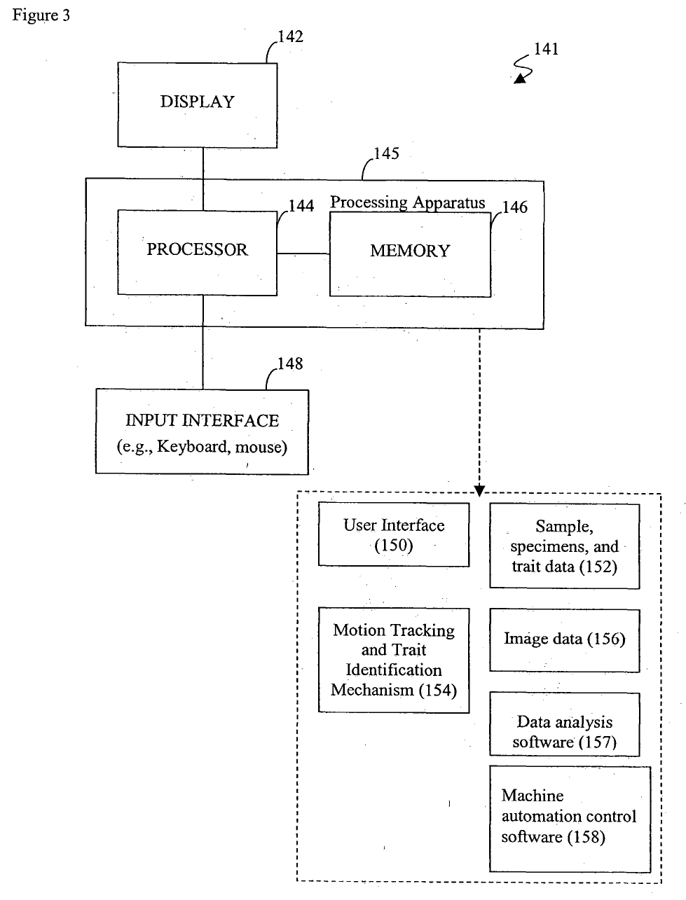

FIG. 3 shows an exemplary assaying computer system 141. A display 142 displays information to the user, including various input and/or output screens and data including, e.g., the motion tracking and trait data. An input interface 148 is provided which comprises a keyboard and a mouse. A processing apparatus 145 is provided which comprises a processor 144 and a memory 146. Collectively, these elements comprises a user interface portion 150, sample, specimen and trait data 152, a motion tracking and trait identification mechanism 154, image data 156, and data analysis software 157, and machine automation control software 158. As used herein “sample data” refers to data corresponding to a particular sample of biological specimens; that is, data which describes the whole sample, such as whether the specimens of the sample are wild-type, mutant, or transgenic, whether the specimens of the sample have been exposed to a candidate agent, sample size, the age and sex of the specimens, the type of specimen in the sample, and the like.

The processing performed by the system shown in processing apparatus 145 may be performed by a general purpose computer alone or in connection with a specialized processing computer. Such processing may be performed by a single platform or by a distributed processing platform. In addition, such processing and functionality can be implemented in the form of special purpose hardware or in the form of software being run by a general purpose processor. Any data handled in such processing or created as a result of such processing can be stored in any memory as is conventional in the art. By way of example, such data may be stored in a temporary memory, such as in the RAM of a given computer system or subsystem. In addition, or in the alternative, such data may be stored in longer-term storage devices, for example, magnetic disks, rewritable optical disks, and so on. For the purposes of the disclosure herein, a computer-readable media (a type of machine-readable media) holding data structures or data may comprise any form of data storage mechanism, including the above-noted types of memory technologies as well as hardware or circuit representations of such data structures and of such data.

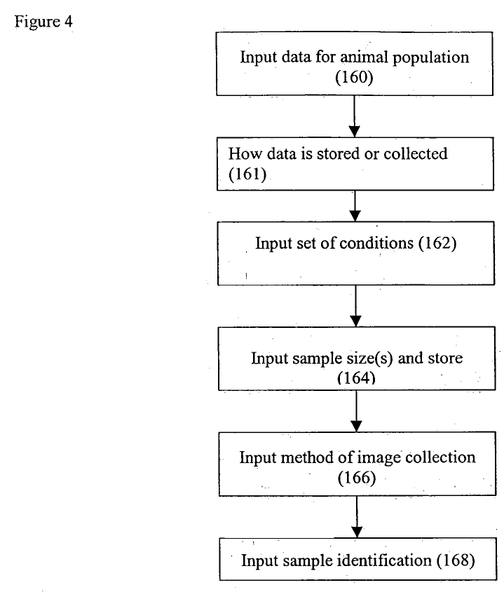

FIG. 4 shows a flowchart of a user interface process performed by the user interface portion 150 shown in FIG. 3. One or more user interface screens are made available to the user on display 142, which have various types of input mechanisms for entering data into a computerized system using input interface 148. In act 160, the user inputs information about the animal population to be assayed; e.g., sample data. Such information may comprise the type of biological specimen (e.g., Drosophila genetically altered by human genes), an identification of a given sample as a reference population, and an identification of another sample as a test population. In act 161, instructions are provided (by default or by input through a user interface) as to how data is to be stored or collected. In act 162, the user inputs a set of conditions defining either specific traits to be determined and stored in the data matrix or a specific central nervous system condition to be studied (which will correspond to a set of traits that will need to be determined and stored in the data matrix), by either choosing a condition from a list and then entering the corresponding set of traits, or by entering the set of traits without choosing a specific condition. Rather than specify the traits or conditions before collecting data, all pertinent data can be collected and stored, and these parameters can be later specified, at the data analysis and/or report or results-display stages, to define the conditions to be assessed and/or the traits to be considered in such assessment.

In act 164, the size of the sample (i.e., “sample data”; the number of specimens per container) is entered by the user. The sample size may be determined by the software automatically (e.g., using the identification mechanism 154 and machine vision techniques to count the specimens per container), or an overridable default number of specimens may be preestablished. In act 166, the method of image collection is input. This may entail specifying the length of time of imaging the sample, and providing instructions regarding different frame rates, different movie lengths, field of view, etc. In act 168, the sample identification is input by the user, which may be a number to specify the sample container 114 being observed.

The movie or series of images of the specimen population is created over the user-specified duration of time, after all the necessary inputs to the user interface are specified and a signal is given by the user, in one embodiment by hitting the Enter key on the keyboard in input interface 148.

Assaying system 141 stores the physical parameter data from the biological specimen population as well as sample data in memory 146. Analysis is performed by analysis software 157 on the physical parameter data from the specimen population in processor 144, and a set of traits may be found to be present in the specimen population.

FIG. 5 shows in schematic form an illustrative embodiment of an assay parameter input screen 180, for setting up the parameters to gather motion-related traits of an biological specimen population in sample containers 114. A specimen information input mechanism 182 allows the user to specify specimen information about the specimen population (e.g., “sample data”), e.g. by using a mouse and a displayed cursor. For example, by clicking on an icon representing specimen information input 182, an input box 184 may be produced that allows the user to choose a specimen population from a list of possible specimen populations, in one embodiment by using the mouse to check a box for the correct biological specimen for both the test population and the reference population. A trait type input mechanism 186 allows the user to specify through a trait set input 188, the traits to be looked at and optionally also whether they relate to a specific central nervous system condition or neurodegenerative disease.

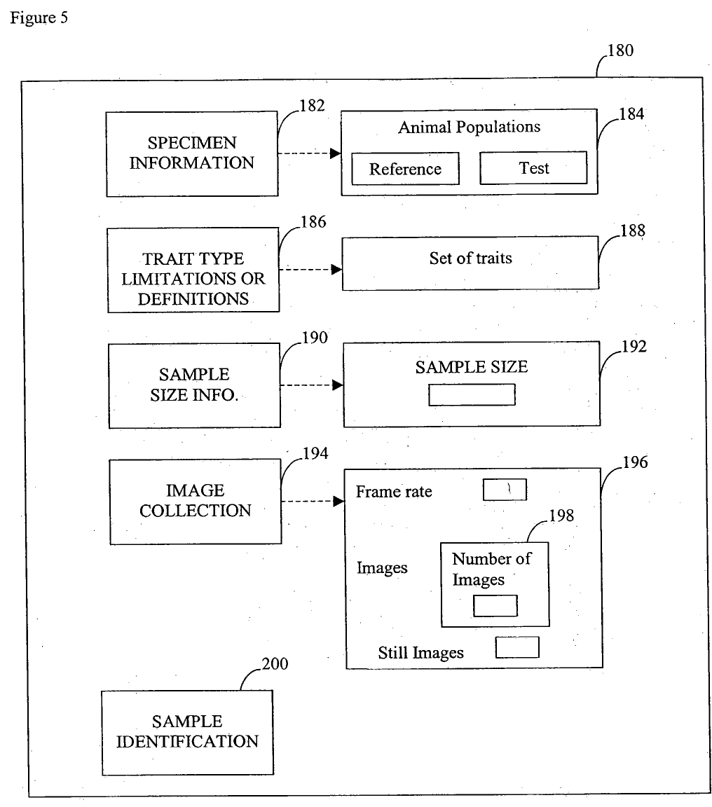

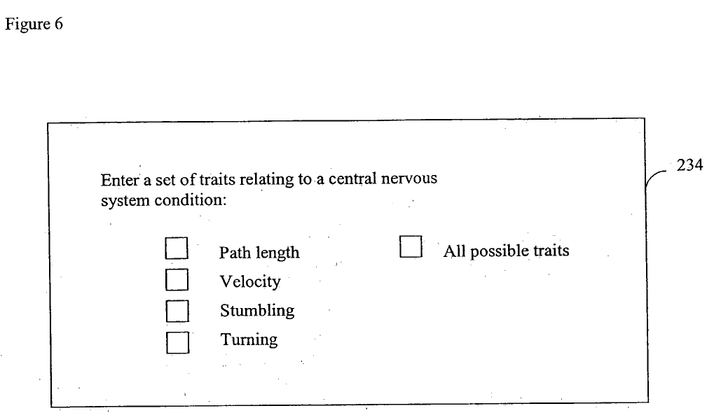

FIG. 6 shows a schematic of a screenshot 234 of the trait set input mechanism 188 in more detail. The user can either enter specific traits to be considered, or choose all traits. Generally, all traits will be acquired and stored during a given assay, and then when analyzing the results, specific traits may be chosen, e.g., using this input screen.

Referring back to FIG. 5, a sample size input mechanism 190 allows the user to specify the sample size. An image collection input mechanism 194 allows the user to specify the way the data is collected and the duration of time of the data collection. The user may use an input box 196, e.g., to specify such parameters as the frame rate, the number of images to be collected, or if still images are to be used. A sample identification input mechanism 200 allows the user to enter an identifier for each sample (vial or container).

Additional features of the computer system may include a comparison mechanism to compare the physical parameter data with a reference physical parameter data set, and an averaging mechanism to average the physical parameter data from a plurality of biological specimen populations in the sample array or from a plurality of specimens within an specimen population (e.g., an animal population).

As noted above, motion tracking apparatus 110 can be used to monitor the activity of an biological specimen population within sample container 114. As also noted above, in one exemplary application, the movement of, for example, flies within sample container 114 can be captured in a movie taken by camera 136, then analyzed by processor 138. As used herein, the term “movie” has its normal meaning in the art and refers a series of images (e.g., digital images) called “frames” captured over a period of time. A movie has two or more frames and usually comprises at least 10 frames, often at least about 20 frames, often at least about 40 frames, and often more than 40 frames. The frames of a movie can be captured over any of a variety of lengths of time such as, for example, at least one second, at least about two, at least about 3, at least about 4, at least about 5, at least about 10, or at least about 15 seconds. The rate of frame capture can also vary. Exemplary frame rates include at least 1 frame per second, at least 5 frames per second or at least 10 frames per second. Faster and slower rates are also contemplated.

The imaging system can identify morphological trait features of the specimens by, for example, capturing still images.

In the present exemplary application, to capture a movie of the movement of flies (although, one of skill in the art could readily adapt the methods taught herein to other biological specimens) within sample container 114, robot 124 grabs a sample container 114 and positions it in front of camera 136. However, before positioning sample container 114 in front of camera 136, robot 124 first raises sample container 114 above a distance, such as about 2 centimeters, above base plate 116, then releases sample container 114, which forces the flies within sample container 114 to fall down to the bottom of sample container 114. Robot 124 then grabs sample container 114 again and positions it to be filmed by camera 136. In one exemplary embodiment, camera 136 captures about 40 consecutive frames at a frame rate of about 10 frames per second. It should be noted, however, that the number of frames captured and the frame rate used can vary. Additionally, the step of dropping sample container 114 prior to filming can be omitted.

As described above, motion tracking apparatus 110 can be configured to receive, store, process, and analyze the movie captured by camera 136. In one exemplary embodiment, processor 138 includes a computer with a frame grabber card configured to digitize the movie captured by camera 136. Alternatively, a digital camera can be used to directly obtain digital images. Motion tracking apparatus 110 can also includes a storage medium 140, such as a hard drive, compact disk, digital videodisc, and the like, to store the digitized movie. It should be noted, however, that motion tracking apparatus 110 can include various hardware and/or software to receive and store the movie captured by camera 136. Additionally, processor 138 and/or storage medium 140 can be configured as a single unit or multiple units.

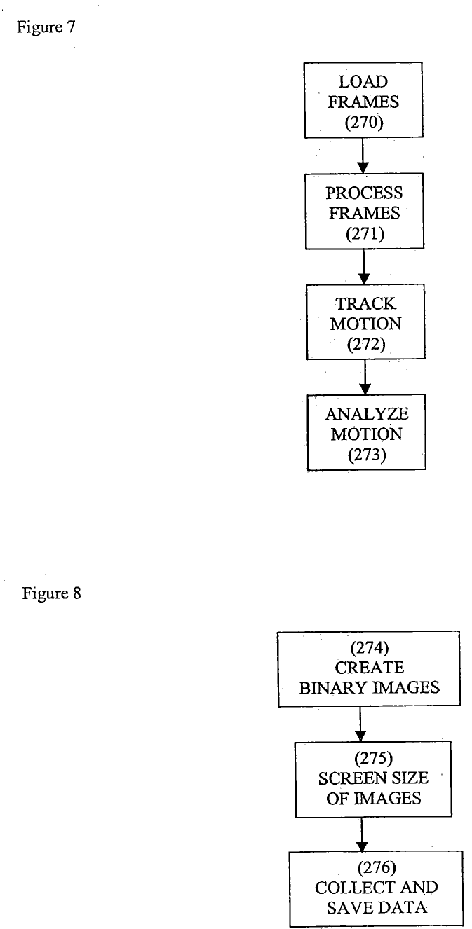

With reference to FIG. 7, an exemplary process of processing and analyzing the movie captured by camera 136 is depicted. In one exemplary embodiment, the exemplary process depicted in FIG. 7 can be implemented in a computer program.

In act 270, the frames of the movie or the series of images are loaded into memory. For example, processor 138 can be configured to obtain one or more frame of the movie from storage medium 140 and load the frames into memory. In act 271, the frames are processed, in part, to identify the specimens within the movie. In act 272, the movements of the specimens in the movie are tracked. In act 273, the movements of the specimens are then analyzed. It should be noted that one or more of these steps can be omitted and that one or more additional steps can also be added. For example, the movements of the specimens in the movie can be tracked (i.e., act 272) without having to analyze the movements (i.e., act 273). As such, in some applications, act 273 can be omitted. In addition, the images can be analyzed while still in RAM, thus eliminating the need for loading of the images.

With reference to FIG. 8, an exemplary process of processing the frames of the movie (i.e., act 271 in FIG. 7) is depicted.

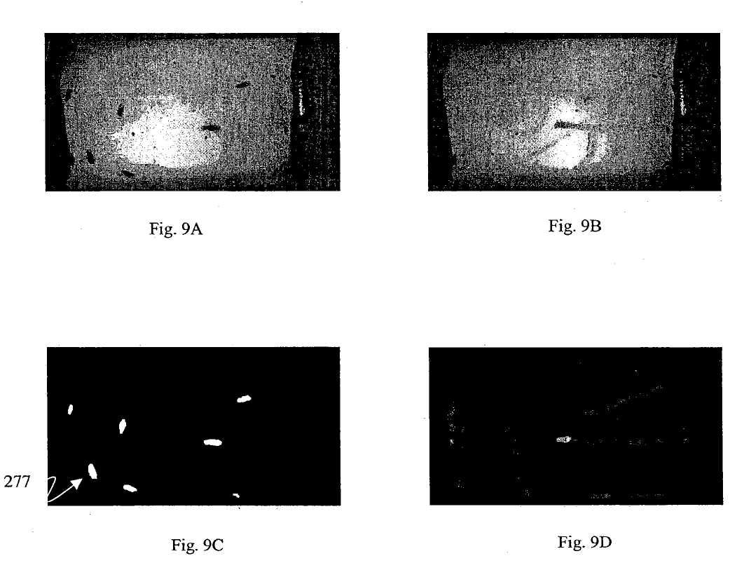

FIG. 9A depicts an exemplary frame of biological specimens within a sample container 114, which in this example are flies within a transparent tube. As used herein, a “biological specimen” refers to an organism of the kingdom Animalia. A “biological specimen”, as used herein may refer to a wild-type specimen, or alternatively, a specimen which comprises one or more mutations, either naturally occurring, or artificially introduced (e.g., a transgenic specimen, or knock-in specimen). A “biological specimen”, as used herein preferably refers to an animal, preferably a non-human animal, preferably a non-human mammal, and can be selected from vertebrates, invertebrates, flies, fish, insects, and nematodes. In one embodiment, a biological specimen is an animal which is no larger in size than a rodent such as a mouse or a rat. Alternatively, a “biological specimen” as used herein refers to an organism which is not a rodent, and more preferably which is not a mouse. In a particularly preferred embodiment, a “biological specimen” as used herein refers to a fly. As used herein, “fly” refers to an insect with wings, such as, but not limited to Drosophila. As used herein, the term “Drosophila” refers to any member of the Drosophilidae family, which include without limitation, Drosophila funebris, Drosophila multispina, Drosophila subfunebris, guttifera species group, Drosophila guttifera, Drosophila albomicans, Drosophila annulipes, Drosophila curviceps, Drosophila formosana, Drosophila hypocausta, Drosophila immigrans, Drosophila keplauana, Drosophila kohkoa, Drosophila nasuta, Drosophila neohypocausta, Drosophila niveifrons, Drosophila pallidiftons, Drosophila pulaua, Drosophila quadrilineata, Drosophila siamana, Drosophila sulfurigaster albostrigata, Drosophila sulfurigaster bilimbata, Drosophila sulfurigaster neonasuta, Drosophila Taxon F, Drosophila Taxon I, Drosophila ustulata, Drosophila melanica, Drosophila paramelanica, Drosophila tsigana, Drosophila daruma, Drosophila polychaeta, quinaria species group, Drosophila falleni, Drosophila nigromaculata, Drosophila palustris, Drosophila phalerata, Drosophila subpalustris, Drosophila eohydei, Drosophila hydei, Drosophila lacertosa, Drosophila robusta, Drosophila sordidula, Drosophila repletoides, Drosophila kanekoi, Drosophila virilis, Drosophila maculinatata, Drosophila ponera, Drosophila ananassae, Drosophila atripex, Drosophila bipectinata, Drosophila ercepeae, Drosophila malerkotliana malerkotliana, Drosophila malerkotliana pallens, Drosophila parabipectinata, Drosophila pseudoananassae pseudoananassae, Drosophila pseudoananassae nigrens, Drosophila varians, Drosophila elegans, Drosophila gunungcola, Drosophila eugracilis, Drosophila ficusphila, Drosophila erecta, Drosophila mauritiana, Drosophila melanogaster, Drosophila orena, Drosophila sechellia, Drosophila simulans, Drosophila teissieri, Drosophila yakuba, Drosophila auraria, Drosophila baimaii, Drosophila barbarae, Drosophila biauraria, Drosophila birchii, Drosophila bocki, Drosophila bocqueti, Drosophila burlai, Drosophila constricta (sensu Chen & Okada), Drosophila jambulina, Drosophila khaoyana, Drosophila kikkawai, Drosophila lacteicornis, Drosophila leontia, Drosophila lini, Drosophila mayri, Drosophila parvula, Drosophila pectinifera, Drosophila punjabiensis, Drosophila quadraria, Drosophila rufa, Drosophila seguyi, Drosophila serrata, Drosophila subauraria, Drosophila tani, Drosophila trapezifrons, Drosophila triauraria, Drosophila truncata, Drosophila vulcana, Drosophila watanabei, Drosophila fuyamai, Drosophila biarmipes, Drosophila mimetica, Drosophila pulchrella, Drosophila suzukii, Drosophila unipectinata, Drosophila lutescens, Drosophila paralutea, Drosophila prostipennis, Drosophila takahashii, Drosophila trilutea, Drosophila bifasciata, Drosophila imaii, Drosophila pseudoobscura, Drosophila saltans, Drosophila sturtevanti, Drosophila nebulosa, Drosophila paulistorum, and Drosophila willistoni. In one embodiment, the fly is Drosophila melanogaster. In the present embodiment, the biological specimen is a fly. As depicted in FIG. 9A, the frame includes images of flies in sample container 114 as well as unwanted images, such as dirt, blemishes, occlusions, and the like. As such, with reference to FIG. 8, in step 274, a binary image is created for each frame of the movie to better identify the images that may correspond to flies in the frames.

In one exemplary embodiment, a background approximation for the movie can be obtained by superimposing two or more frames of the movie, then determining a characteristic pixel value for the pixels in the frames. A characteristic pixel value as used herein refers to an average pixel value for a given area of a given frame, and may be determined using, for example, average pixel value, a median pixel value, and the like. Additionally, the background approximation can be obtained based on a subset of frames or all of the frames of the movie. The background approximation normalizes non-moving elements in the frames of the movie. FIG. 9B depicts an exemplary background approximation. In the exemplary background approximation, note that the fly images in FIG. 9A have been removed, so that subtracting the remaining approximation from the original only leaves moving flies.

To generate a binary image, the background approximation is subtracted from a frame of the movie. By subtracting the background approximation from a frame, the binary image of the frame captures the moving elements of the frame. Additionally, a gray-scale threshold can be applied to the frames of the movie. For example, if a pixel in a frame is darker than the threshold, it is represented as being white in the binary image. If a pixel in the frame is lighter than the threshold, it is represented as being black in the binary image. More particularly, if the difference between an image pixel value and the background pixel value is less than the difference between a threshold value and the value of a white pixel (i.e., [Image Pixel Value]−[Background Pixel Value]<[Threshold Value]−[Pixel Value of White Pixel]), then the binary image pixel is set as white. For example, if the pixel value of a black pixel is assumed to be 0 and a white pixel is assumed to be 255, an exemplary threshold value of 230 can be used.

With reference again to FIG. 8, in step 275, the image blocks in the frames of the movie are screened by pixel size. More particularly, image blocks in a frame having an area greater than a maximum threshold or less than a minimum threshold are removed from the binary image. For example, FIG. 9C depicts an exemplary binary image, which was obtained by subtracting the background approximation depicted in FIG. 9B from the exemplary frame depicted in FIG. 9A and removing image blocks in the frames having areas greater than 1600 pixels or less than 30 pixels. The image blocks are also screened for eccentricity. As used herein, “eccentricity” refers to the relationship between width and length of an image block. For example, where a biological specimen of the invention is a fly, the accepted eccentricity values range between 1 and 5 (that is, the ratio of width to length is within a range of 1 to 5). The eccentricity value of a given biological specimen can be determined empirically by one of skill in the art based on the average width and length measurements of the specimen. Once the eccentricity value of a given biological specimen is determined, that value will be permitted to increase by a doubling of the value or decrease by half the value, and still be considered to be within the acceptable range of eccentricity values for the particular biological specimen. Image blocks which fall outside the accepted eccentricity value for a given biological specimen (or sample of plural biological specimens) will be excluded from the analysis (i.e., blocks that are too long and/or narrow to be a fly are excluded).

As depicted in FIG. 9C, the image blocks 277 that may correspond to specimens, and more specifically flies in this present exemplary application, can be more easily identified in the binary image. FIG. 9D depicts a normalized sum of the binary images of the frames of the movie, which can provide an indication of the movements of the flies during the movie. In FIGS. 9C and 9D, image blocks 277 are depicted as being white, and the background depicted as being black. It should be noted, however, that image blocks 277 can be black, and the background white.

With reference to FIG. 8, in step 276, data on image blocks 277 (FIG. 9C) are collected and stored. In one exemplary embodiment, the collected and stored data can include one or more characteristics of image blocks 277 (FIG. 9C), such as length, width, location of the center, area, and orientation.

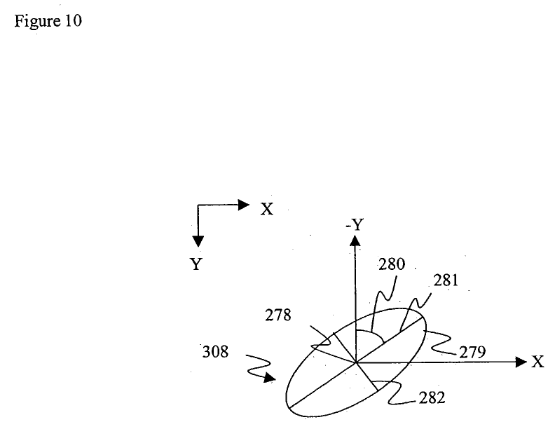

With reference to FIG. 10, a long axis 281 and a short axis 282 for image block 277 can be determined based on the shape and geometry of image block 277. The length of long axis 281 and the length of short axis 282 are stored as the length and width, respectively, of image block 277.

A center 278 can be determined based on the center of gravity of the pixels for image block 277. The center of gravity can be determined using the image moment for an image block 277, according to methods which are well established in the art. The location of center 278 can then be determined based on a coordinate system for the frame. With reference to FIG. 2, in the present example, camera 136 is tilted such that the frames captured by camera 136 are rotated 90 degrees. As such, as indicated by the coordinate system used in FIG. 10, in the frames captured by camera 136, the top and bottom of sample container 114 is located on the left and right sides, respectively, of the frame. Furthermore, as indicated by the coordinate system used in FIG. 10, for the purpose of tracking the movement of image blocks 277, the X-axis corresponds to the length of sample container 114, where the zero X position corresponds to a location near the top of sample container 114. The Y-axis corresponds to the width of sample container 114, where the zero Y position corresponds to a location near the right edge of sample container 114 as depicted in FIG. 2A. Thus, when a fly moves from the bottom of sample container 114 toward the top, it moves in a negative X direction. When the fly moves from left to right in the sample container 114, it moves in a negative Y direction. In one exemplary embodiment, the zero X and Y position is the upper left corner of a frame. It should be noted that the labeling of the X and Y axes is arbitrary and provided for the sake of convenience and clarity.

With reference to FIG. 10, an area 279 can be determined based on the shape and geometry of image block 277. For example, area 279 can be defined as the number of pixels that fall within the bounds of image block 277. It should be noted that area 279 can be determined in various manners and defined in various units.

An orientation 280 can be determined based on long axis 281 for image block 277. For example, as depicted in FIG. 10, orientation 280 can be defined as an angle long axis 281 of image block 277 and an axis of the coordinate system of the frame, such as the Y axis as depicted in FIG. 10. It should be noted that orientation 280 can be determined and defined in various manners.

In one exemplary embodiment, data for image blocks 277 in each frame of the movie are first collected and stored. As described below, trajectories of the image blocks 277 are then determined for the entire movie. Alternatively, data for image blocks 277 and the trajectories of the image blocks 277 can be determined frame-by-frame.

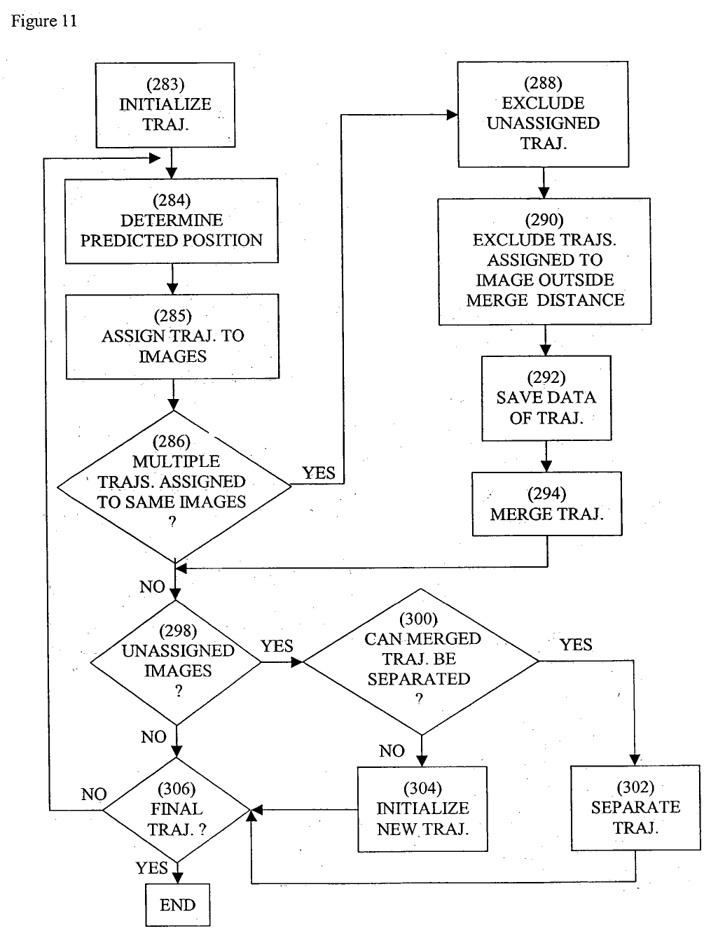

With reference to FIG. 7, in the present embodiment, in step 272, the movements of the specimens in the movie are tracked. More particularly, FIG. 11 depicts an exemplary process for tracking the movements of the specimens in the movie or series of images. In one exemplary embodiment, the exemplary process depicted in FIG. 11 can be implemented in a computer program.

In act 283, for the first frame of the movie, trajectories of image blocks 277 (FIG. 9C) are initialized. More specifically, a trajectory is initialized for each image block 277 identified in the first frame. The trajectory includes various data, such as the location of the center, area, and orientation of image block 277. The trajectory also includes a velocity vector, which is initially set to zero.

In act 284, a predicted position is determined. For example, the predicted position of an image block 277 (FIG. 9C) and/or trajectory can be determined based on its previous position and velocity vector. More specifically, in one configuration, the predicted position can be determined as: [Predicted Position]=[Previous Position]+[Prediction Factor]×[Previous Velocity Vector], where the prediction factor can vary between zero and one, and may be empirically determined by one of skill in the art.

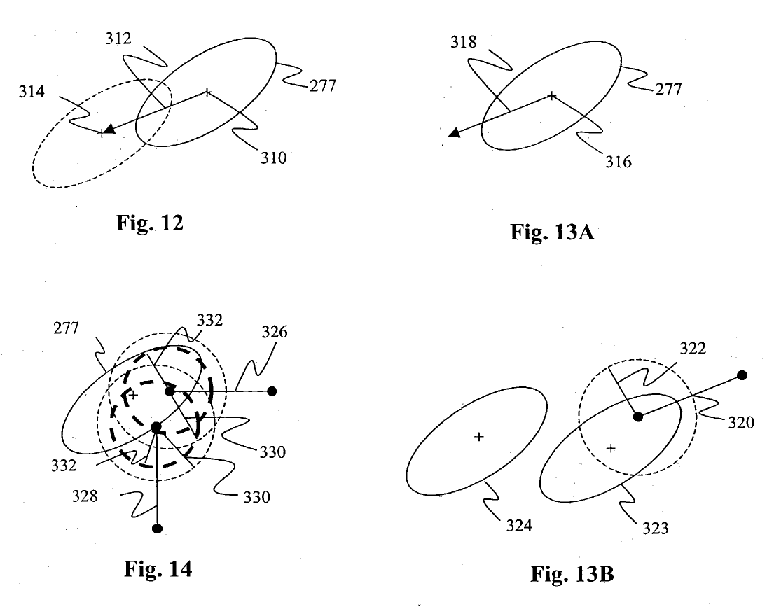

For example, with reference to FIG. 12, assume that in one frame a trajectory having a center position 310 and a velocity vector 312 has been initialized based on image block 277. If the prediction factor is zero, the predicted position in the next frame would be the previous center position 310. If the prediction factor is one, the prediction position in the next frame would be position 314. In one exemplary embodiment, a prediction factor of zero is used, such that the predicted position is the same as the previous position. However, the prediction factor used can be adjusted and varied depending on the particular application.

Additionally, a predicted velocity can be determined based on the previous velocity vector. For example, the predicted velocity can be determined to be the same as the previous velocity.

With reference to FIG. 11, in act 285, the next frame of the movie is loaded and the trajectories are assigned to image blocks 277 (FIG. 9C) in the new frame. More specifically, each trajectory of a previous frame is compared to each image block 277 (FIG. 9C) in the new frame. If only one image block 277 (FIG. 9C) is within a search distance of a trajectory, and more specifically within the predicted position of the trajectory, then that image block 277 (FIG. 9C) is assigned to that trajectory. If none of the image blocks 277 (FIG. 9C) are within the search distance of a trajectory, that trajectory is unassigned and will be hereafter referred to as an “unassigned trajectory.” However, if more than one image block 277 (FIG. 9C) falls within the search distance of a trajectory, and more specifically within the predicted position of the trajectory, the image block 277 (FIG. 9C) closest to the predicted position of that trajectory is assigned to the trajectory.

For example, in one exemplary embodiment, if more than one image block 277 (FIG. 9C) falls within the search distance of a trajectory, a distance between each of the image blocks 277 (FIG. 9C) and the trajectory can be determined based on the position of the image block 277 (FIG. 9C), the prediction position of the trajectory, a speed factor, the velocity of the image block 277 (FIG. 9C), and the predicted velocity of the trajectory. More particularly, the distance between each image block 277 (FIG. 9C) and the trajectory can be determined as the value of: norm([Position of the image block]−[Predicted position of the image block]+[Speed factor]*norm([Velocity]−[Predicted Velocity])). A norm function is the length of a two-dimensional vector, meaning that only the magnitude of a vector is used. The speed factor can be varied from zero to one, where zero corresponds to ignoring the velocity of the image block and one corresponds to giving equal weight to the velocity and the position of the image block. In the present exemplary embodiment, the image block 277 (FIG. 9C) having the shortest distance is assigned to the trajectory. Additionally, a speed factor of 0.5 is used.

With reference to FIG. 13A, assume that in one frame a trajectory having a center position 316 and a velocity vector 318 has been initialized based on image block 277. With reference to FIG. 13B, in the next frame, the trajectory, which is now depicted as trajectory 320, is assigned to an image block 277. Assuming that a prediction factor of zero is used, a search distance 322 associated with trajectory 320 is centered about the previous center position 316 (FIG. 13A). Thus, in the example depicted in FIG. 13B, image block 323 is assigned to trajectory 320, while image block 324 is not. In one exemplary embodiment, a search distance of [350 pixels per second]/[frame rate] is used, where the frame rate is the frame rate of the movie. For example, if the frame rate is 5 frames per second, then the search distance is 70 pixels/frame. It should be noted that various search distances can be used depending on the application.

With reference to FIG. 11, in act 286, the trajectories of the current frame are examined to determine if multiple trajectories have been assigned to the same image block 277 (FIG. 9C). For example, with reference to FIG. 14, assume that image block 277 lies within search distance 330 of trajectories 326 and 328. As such, image block 277 is assigned to trajectories 326 and 328.

With reference to FIG. 11, in act 288, unassigned trajectories are excluded from being merged. More particularly, multiple trajectories assigned to an image block 277 (FIG. 9C) are examined to determined if any of the trajectories were unassigned trajectories in the previous frame. The unassigned trajectories are then excluded from being merged.

In act 290, trajectories assigned to an image block 277 outside of a merge distance are excluded from being merged. For example, with reference to FIG. 14, assume that a merge distance 332 is associated with trajectories 326 and 328. If image block 277 does not lie within merge distance 332 of trajectories 326 and 328, the two trajectories are excluded from being merged. If image block 277 does lie within merge distance 332 of trajectories 326 and 328, the two trajectories are merged. In one exemplary embodiment, a merge distance of [250 pixels per second]/[frame rate] is used. As such, if the frame rate if 5 frames per second, then the merge distance is 50 pixels/frame.

One of skill in the art will appreciate that a separation distance, merge distance, and search distance used in the methods of the invention may be modified depending on the particular biological specimen to be analyzed, frame rate, image magnification, and the like. In emperically determining a search, merge, and separation distance for a given biological specimen, one of skill in the art will appreciate that the value used is based on an anticipated distance which a specimen will move between frames of the movie, and will also vary with the size of the specimen, and the speed at which the frames of the movie are acquired.

With reference to FIG. 11, in act 292, for trajectories that were not excluded in acts 288 and 290, data for the trajectories are saved. More particularly, an indication that the trajectories are merged is stored. Additionally, one or more characteristics of the image blocks 277 (FIG. 14) associated with the trajectories before being merged is saved, such as area, orientation, and/or velocity. As described below, this data can be later used to separate the trajectories. In act 294, the multiple trajectories are then merged, meaning that the merged trajectories are assigned to the common image block 277 (FIG. 14).

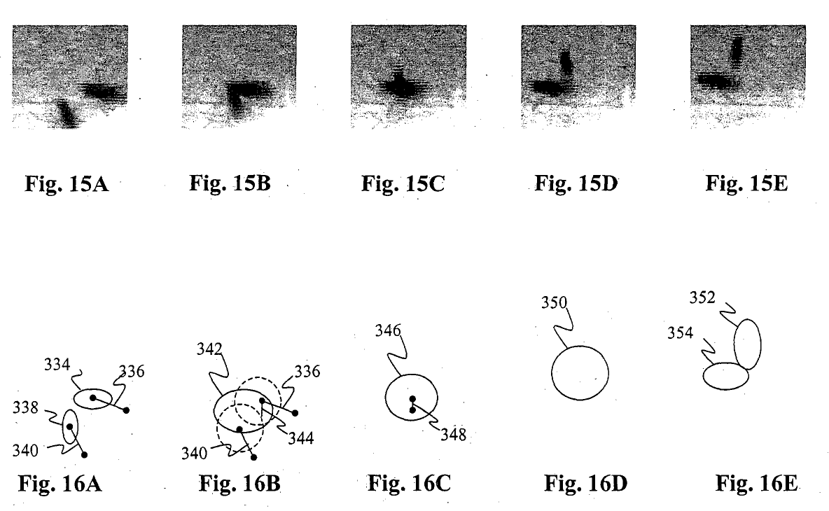

For example, FIGS. 15A to 15C depict three frames of a movie where two flies converge. Assume that FIGS. 16A to 16C depict binary images of the frames depicted in FIGS. 15A to 15C, respectively. While these figures specifically show the movements of flies, the methods of the invention may be readily adapted to monitor the trajectories and thus the physical trait data of other non-fly biological specimens.

In FIG. 16A, two image blocks 334 and 338 are identified, which correspond to the two flies depicted in FIG. 15A. Assume that trajectories 336 and 340 were assigned to image blocks 334 and 338, respectively, in a previous frame. As such, the data for trajectory 336 includes characteristics of image block 334, such as area, orientation, and/or velocity. Similarly, the data for trajectory 340 includes characteristics of image block 338, such as area, orientation, and/or velocity.

As depicted in FIG. 16B, assume that the two flies depicted in FIG. 15B are in sufficient proximity that in the binary image of the frame that a single image block 342 is identified. As also depicted in FIG. 16B, image block 342 lies within search distance 344 of trajectories 336 and 340. As such, image block 342 is assigned to trajectories 336 and 340. Additionally, assume that image block 342 falls within the merge distance of trajectories 336 and 340. As such, in accordance with act 292 (FIG. 11), data for trajectories 336 and 340 are saved. More specifically, one or more characteristics of image blocks 334 and 338 (FIG. 16A) are stored for trajectories 336 and 340, respectively. In accordance with act 294 (FIG. 11), trajectories 336 and 340 are merged, meaning that they are associated with image block 342.

As depicted in FIG. 16C, assume that the two flies depicted in FIG. 15C remain in sufficient proximity that in the binary image of the frame that a single image block 346 is identified. As such, trajectories 336 and 340 (FIG. 16B) remain merged. As also depicted in FIG. 16C, image block 346 can have a different shape, area, and orientation than image block 342 in FIG. 16B. Now assume that velocity vector 348 is calculated based on the change in the position of the center of image block 346 from the position of the center of image block 342 (FIG. 15B). As such, the data of the trajectory of image block 346 is appropriately updated.

Although in the above example two trajectories corresponding to two flies are merged, it should be noted that any number of trajectories corresponding to any number of flies can be merged. For example, rather than two flies crossing paths as depicted in FIGS. 15A to 15C, three or more flies can converge.

As noted above, with reference again to FIG. 11, in act 290, trajectories that are determined to have been unassigned trajectories in the previous frame are excluded from being merged with other trajectories. For example, with reference to FIG. 14, if trajectory 328 is determined to have been an unassigned trajectory in the previous frame, meaning that it had not been assigned to any image block 277 (FIG. 9C) in the previous frame, then trajectory 328 is not merged with trajectory 326. Instead, in one embodiment, trajectory 326 is assigned to image block 277 (FIG. 9C), while trajectory 328 remains unassigned.

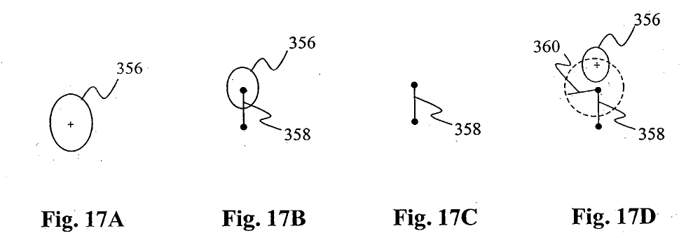

Now assume that FIGS. 17A to 17E depict the movement of a fly over five frames of a movie. More specifically, assume that during the five frames the fly begins to move, comes to a stop, and then moves again.

Assume FIG. 17A depicts the first frame. As such, a trajectory corresponding to image block 356 is initialized. As depicted in FIG. 17B, assume that the fly has moved and that image block 356 is the only image block that falls within the search distance of the trajectory that was initialized based on image block 356 in the earlier frame depicted in FIG. 17A. As such, trajectory 358 is assigned to image block 356 and the data for trajectory 358 is updated with the new location of the center, area, and orientation of image block 356. Additionally, a velocity vector is calculated based on the change in location of the center of image block 356.

Now assume that the fly comes to a stop. As described above, in one exemplary embodiment, a background approximation is calculated and subtracted from each frame of the movie. As also described above, flies that do not move throughout the movie are averaged out with the background approximation. As such, when a fly comes to a stop, the image block of that fly will decrease in area. Indeed, if the fly remains stopped, the image block can decrease until it disappears. Additionally, a fly can also physically leave the frame.

As depicted in FIG. 17C, assume in the present example that the fly has remained stopped sufficiently long enough that image block 356 (FIG. 17B) has disappeared in the present frame. As such, trajectory 358 becomes an unassigned trajectory.

Now assume that the fly begins to move again. As such, as depicted in FIG. 17D, image block 356 is identified. Now assume that the area of image block 356 is sufficiently large that image block 356 lies within search distance 360 of trajectory 358. As such, trajectory 358 now becomes assigned to image block 356.

With reference now to FIG. 11, in act 298, image blocks 277 (FIG. 9C) in the current frame are examined to determine if any remain unassigned. In act 300, the unassigned image blocks are used to determine if any merged trajectories can be separated. More specifically, if an unassigned image block falls within a separation distance of a merged trajectory, one or more characteristics of the unassigned image block is compared with one or more characteristics that were stored for the trajectories prior to the trajectories being merged to determine if any of the trajectories can be separated from the merged trajectory.

For example, in one exemplary embodiment, the area of the unassigned image block can be compared to the areas of the image blocks associated with the trajectories before the trajectories were merged. As described above, this data was stored before the trajectories were merged. The trajectory with the stored area closest to the area of the unassigned image block can be separated from the merged trajectory and assigned to the unassigned image block. Alternatively, if the stored area of a trajectory and that of the unassigned image block are within a difference threshold, then that trajectory can be separated from the merged trajectory and assigned to the unassigned image block.

It should be noted that orientation or velocity can be used to separate trajectories. Additionally, a combination of characteristics can be used to separate trajectories. Furthermore, if a combination of characteristics is used, then a weight can be assigned to each characteristic. For example, if a combination of area and orientation is used, the area can be assigned a greater weight than the orientation.

As described above, FIGS. 15A to 15C depict three frames of a movie where two flies converge, and FIGS. 16A to 16C depict binary images of the frames depicted in FIGS. 15A to 15C. Similarly, FIGS. 15D and 15E depict two frames of the movie where the two flies diverge, and FIGS. 16D and 16E depict binary images of the frames depicted in FIGS. 15D and 15E.

As described above, a merged trajectory was created based on the merging of image blocks 334 and 338 (FIG. 16A) into image blocks 342 (FIG. 16B) and 346 (FIG. 16C). Assume that in FIG. 16D, the merged trajectories remain merged for image block 350. However, in FIG. 16E, assume that the flies have separated sufficiently that an image block 352 is identified apart from image block 354. Additionally, assume that in the frame depicted in FIG. 16E image block 352 is not assigned to a trajectory, but falls within the separation distance of the merged trajectory. As such, in accordance with act 300, one or more characteristics of image block 352 is compared with the stored data of the merged trajectories. More specifically, in accordance with the exemplary embodiment described above, the area of image block 352 is compared with the stored areas of image blocks 334 and 338 (FIG. 16A), which correspond to the image blocks that were associated with trajectories 336 and 340 (FIG. 16B), respectively, before the trajectories were merged. In this example, the stored area image block 338 (FIG. 16A), which corresponds to trajectory 340 (FIG. 16B) before it was merged with trajectory 336 (FIG. 16B), most closely matches the area of image block 352. As such, trajectory 340 (FIG. 16B) is separated from the merged trajectory and assigned to image block 352.

With reference again to FIG. 11, in act 304, if an unassigned image block does not fall within the separation distance of any merged trajectory, then a new trajectory is initialized for the unassigned image blocks. In one embodiment, a separation distance of 300/[frame rate], where the frame rate is the frame rate of the movie, is used. It should be noted, however, that various separation distances can be used.

In act 306, if the final frame has not been reached, then the motion tracking process loops to act 284 and the next frame is processed. If the final frame has been reached, then the motion tracking process is ended.

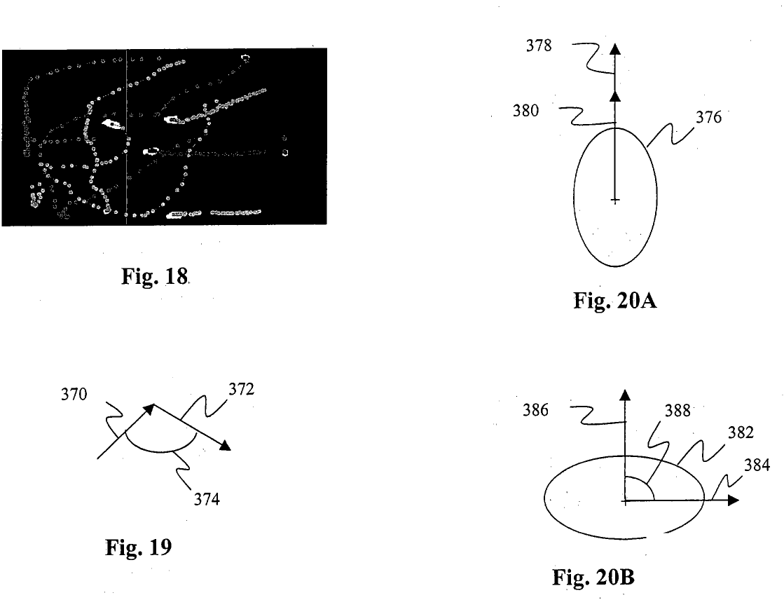

In this manner, with reference to FIG. 2, the movements of the flies within sample container 114 as captured by camera 136 can be processed. For example, FIG. 18 depicts the trajectories of the flies depicted in FIG. 9A.

Having thus tracked the movements of the specimens within sample container 114, the movements can then be analyzed for various characteristics and/or traits. For example, in one embodiment, various statistics on the movements of the specimens, such as the x and y travel distance, path length, speed, turning, and stumbling, can be calculated. These statistics can be determined for each trajectory and/or averaged for a population, such as for all the specimens in a sample container 114).

In the present embodiment, x and y travel distances can be determined based on the tracked positions of the centers of image blocks 277 (FIG. 9C) and/or the velocity vectors of the trajectories. As noted above, the x and y travel distance for each trajectory can be determined, which can indicate the x and y travel distance of each specimen within sample container 114. Additionally or alternatively, an average x and y travel distance for a population, such as all the specimens in a sample container 114, can be determined.

Path length can also be determined based on the tracked positions of the centers of image blocks 277 (FIG. 9C) and/or the velocity vectors of the trajectories. Again, a path length for each trajectory can be determined, which can indicate the path length for each specimen within sample container 114. Additionally or alternatively, an average path length for a population, such as all the specimens in a sample container 114, can be determined.

Speed can be determined based on the velocity vectors of the trajectories. An average velocity for each trajectory can be determined, which can indicate the average speed for each specimen within sample container 114. Additionally or alternatively, an average speed for a population, such as all the specimens in a sample container 114, can be determined.

Turning can be determined as the angle between two velocity vectors of the trajectories. For example, with reference to FIG. 19, assume that velocity vector 370 was determined based on the movement of a specimen between frames 1 and 2; and velocity vector 372 was determined based on the movement of the specimen between frames 2 and 3. As such, in this example, angle 374 defines the amount of turning captured in frames 1, 2, and 3. In this manner, the amount of turning for each trajectory can be determined, which can indicate the amount of turning for each specimen within sample container 114. As used herein, “turning” refers to a change in the direction of the trajectory of a specimen such that a second trajectory is different from a first trajectory. Turning may be determined by detecting the existence of an angle 374 between the velocity vector of a first frame and a second frame. More specifically, “turning” may be determined herein as an angle 374 of at least 1°, preferably greater than 2°, 5°, 10°, 20°, 30′, 40°, 50°, and up to or greater than 90°. Additionally or alternatively, an average amount of turning for a population, such as all the specimens in a sample container 114, can be determined.

Stumbling can be determined as the angle between the orientation of a image block 277 (FIG. 9C) and the velocity vector of the image block 277 (FIG. 9C) of the trajectories. For example, with reference to FIG. 20A, assume that orientation 378 and velocity vector 380 of an image block 376 of a trajectory are aligned (i.e., the angle between orientation 378 and velocity vector 380 is zero degrees). As such, in this instance, the amount of stumbling is zero, and thus at a minimum. With reference to FIG. 20B, now assume that orientation 384 and velocity vector 386 of image block 382 of a trajectory are perpendicular (i.e., the angle between orientation 384 and velocity vector 386 is 90 degrees). As such, in this instance, amount of stumbling defined by angle 388 is 90 degrees, and thus at a maximum. In this manner, the amount of stumbling for each trajectory can be determined, which can indicate the amount of stumbling for each specimen within sample container 114. Accordingly, “stumbling” as used herein refers to a difference between the direction of the orientation vector and the velocity vector of a biological specimen. “Stumbling” may be determined according to the invention, by the presence of an angle between the orientation vector and velocity vector of a biological specimen of at least 1°, preferably greater than 2°, 5°, 10°, 20°, 40°, 60°, and up to or greater than 90°. Additionally or alternatively, an average amount of stumbling for a population, such as all the specimens in a sample container 114, can be determined.

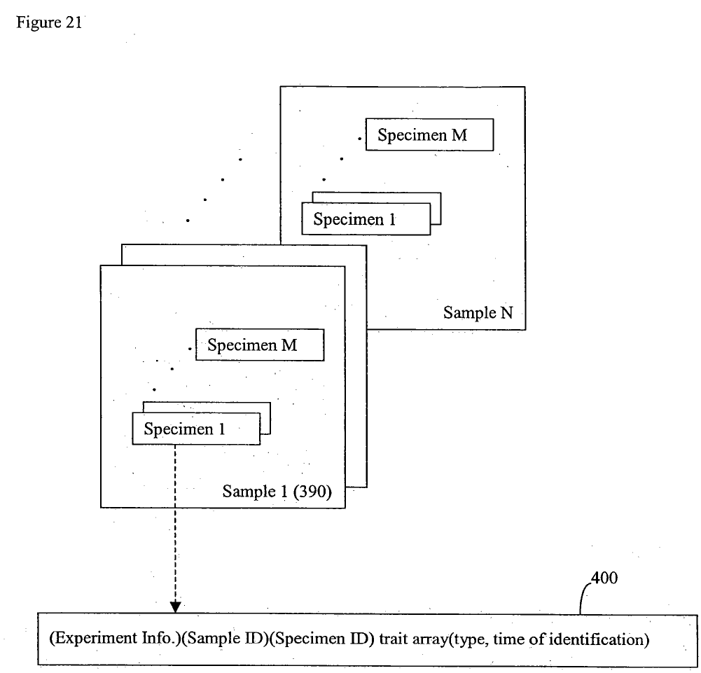

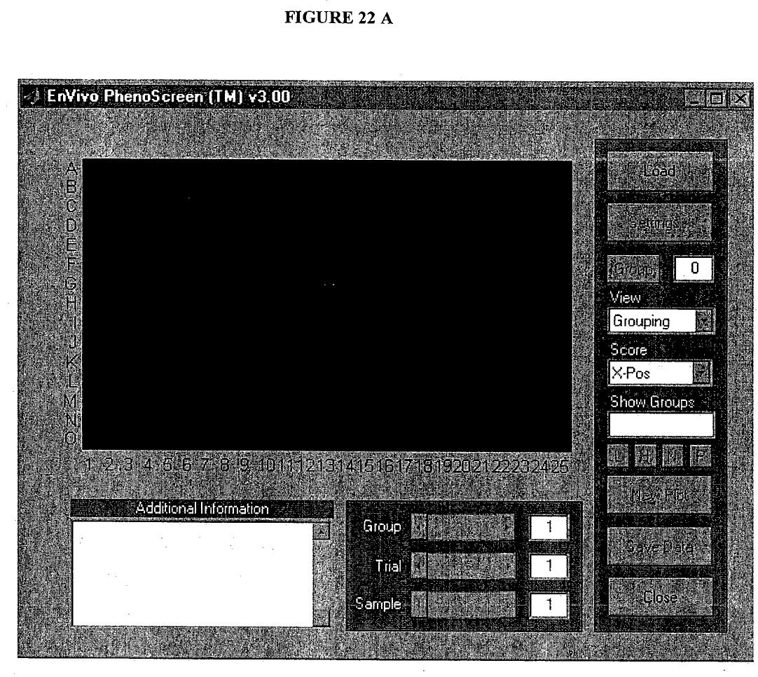

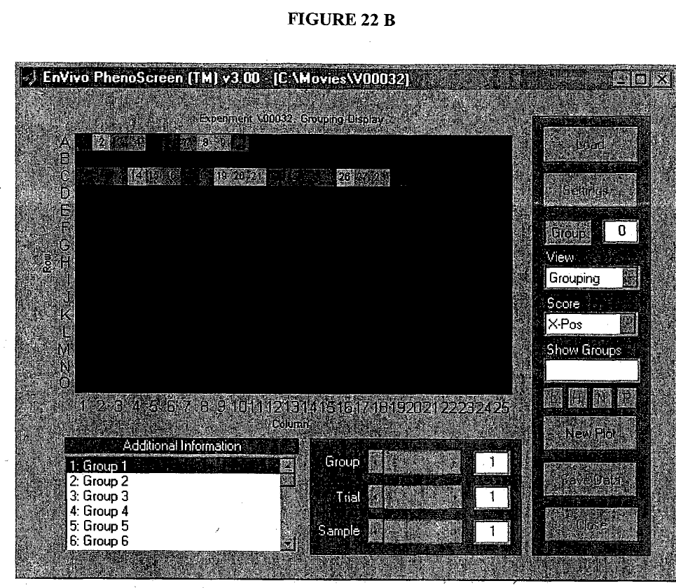

The results of the motion tracking algorithm are displayed in a data matrix as shown in FIG. 21. The data matrix consists of a data array for each sample. Within each data array is a specimen data array for each specimen within the sample. For example, data array 390 is for sample 1. The sample identification number and specimen identification number are displayed, along with the motion traits that each specimen within the animal population exhibited in data box 400 for each specimen within the sample. The motion traits can be a simple listing, or can be broken up by time, showing the motion trait in each designated block of time.

Data Analysis Software—A Specific Embodiment.

Software may be designed to analyze the raw data collected from an assay system. In this embodiment, such software comprises a user interface to manipulate, group, and view the analyzed or “tracked” data. Companion automation control software may be provided to run the assay machine. It will be appreciated by one of skill in the art, that while the specific examples below refer to embodiments wherein a sample comprises specimens which are flies, the methods described herein are adaptable to the analysis of a sample in which the specimen is not a fly but is another, different type of biological specimen.

Start View.

FIG. 22A illustrates a window that comes up when the program is initiated. The black section demarcates the representation of the screening machine's deck. The illustrated machine can accommodate 375 vials (15×25) designated by location with row letters A to 0 and column numbers 1 to 25. The top left corner is therefore vial “A01”. The “Load” button is used to open an experiment. When pressed for the first time for an experiment, the vials of that experiment will be automatically grouped into one group per vial and given default names, as is shown in the example experiment V00032 shown in FIG. 22B. Proper default values will be set for all parameters and the program will automatically go to the grouping view, from where grouping as well as group and vial properties can be altered. Experiments can be simultaneously tracked as soon as an assay has been initiated on the assay machine. The “Settings” button provides the user the freedom of changing certain default options (e.g. Error bar calculation, trial or repeats used for analysis, statistics, etc.). The “Group” button is used to view the data based on defined groups of vials. The “Show Groups” edit box is used when viewing more than one group at the same time. The small buttons below are used for plot formatting purposes.

Grouping View.

In the grouping view one can set up how the groups are composed, assign names to groups, and compensate for varying number of flies in the vials. Groups are assigned by entering the group number desired to assign in the edit box to the right of the “Group” button, and then left-clicking on the vial position to assign to that group. To allow for faster grouping of vials, it is also possible to right-click somewhere over the grouping display, in which case the number of the current group will be incremented by one. Furthermore, the group number zero has a special meaning and denotes a dummy group which will be excluded from all analysis. The vials excluded in this way are marked in the grouping view with a gray color and the symbol “-”, whereas for all other vials their group colors and numbers are shown.

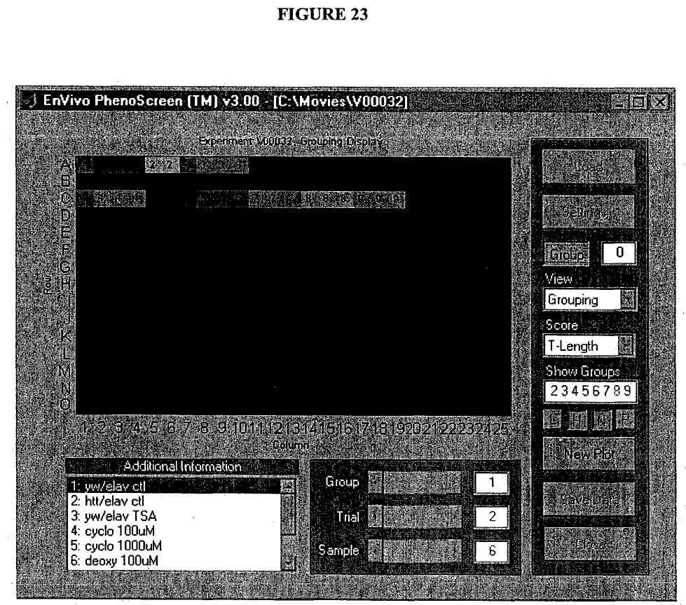

FIG. 23 shows an example for V00032, where three vials have been used in each group, except the empty vials at A01 and C01 and an erroneous vial at A07, which have been excluded.

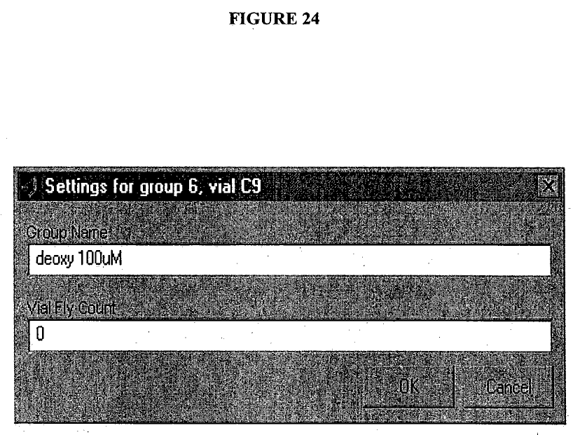

FIG. 24 shows a dialog box produced by double-clicking on one of the vial positions to set a few additional parameters for that group and vial. The group name field allows one to set a name for that group number which will then be used in the other views. By entering names, one can thereby avoid keeping track of which group number was associated with which treatment. Moreover, the vial fly count field is used to override the default fly count in the settings dialog (see next section). It will be recognized by one of skill in the art that the value to be entered in “vial fly count” will be the number of any type of specimens in a sample, and is thus not limited to analyses where the specimen is a fly. The scores affected by the number of flies (or specimen) in the vial will then be accordingly compensated. Zero is a special value indicating that the default fly count should be used for this vial, and initially all vials have this value. Entering nothing will render the same thing. In the example to the left above, one can see the names assigned to groups in the additional information box. Last, one can use the “Group” button regardless of which view the user is looking at, because the last view is remembered by the program (i.e., pressing it will bring one to the grouping display and let the user modify the grouping). Releasing it will then bring the user back to the previous view. Settings Dialog.

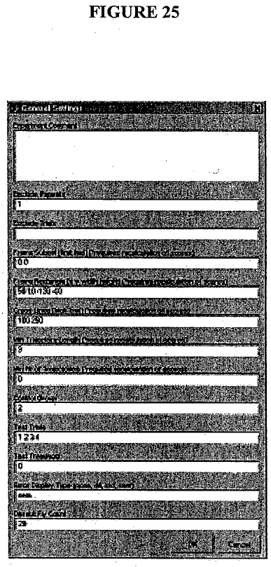

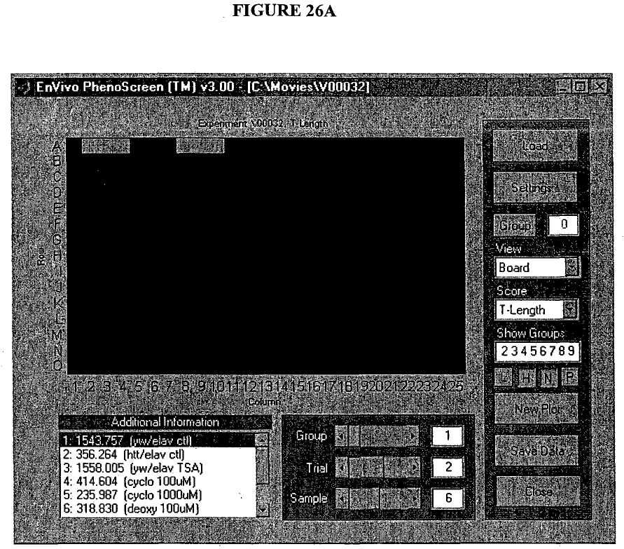

FIG. 25 shows a dialog window, produced by clicking on the “Settings” button. In this dialog window, the general settings of the analysis program can be changed. Changing one or more of the fields marked with an asterisk will require scores to be recalculated, which will take some minute or so after the OK button has been pressed. Entering erroneous values and pressing OK will result in the box being redisplayed with an error message in the title bar. The first field is simply the experiment comment from the assay machine control program, which can also be changed.

“Exclude Repeats” lets the user exclude repeats from the analysis by entering the repeat numbers separated by spaces. Entering nothing includes all repeats. “Exclude Trials” works in the same way, but is used to exclude entire trials instead. This will also prevent them from being displayed in the plots. “Frame Subset” lets one enter two numbers denoting the first and the last frame of a range to be used. Entering two zeros or nothing will include all frames. The last number can be negative to instead give distance in number of frames from the end of the movie. The frame range currently used is showed in the sample view. “Frame Rectangle” is used to only include data that is inside a certain rectangle of the entire frame. The width and height values can be negative to indicate distance from the right and bottom edges of the movie, respectively. The frame rectangle is shown in the sample view. “Cross Lines” sets the two x-coordinates used for calculating the high and low cross scores found in the score dropdown box (previously called Cross150 and Cross250). Also these two lines are shown in the sample view.

With “Min Trajectory Length” one can require the trajectory to be of a certain length for it not to be excluded. For example, setting this value to 3 will remove all trajectories consisting of only 1 or 2 points from consideration. (Often when flies fly around in the vial that gives rise to one- or two-point trajectories.) Similarly, “Min Nr of Trajectories” requires at least that number of trajectories to be detected for a movie for that movie to be used. Setting any of the last two values to zero or empty turns that feature off. Entering a group number in “Control Group” will allow one to perform statistical comparisons to that group in the board view.

The trials the user wants to perform the analysis on are entered in “Test Trials”. The measure seen in the board view when having set these two fields will be the average difference from the control group in number of standard deviations, i.e. the test trials should be set to the trial numbers where one expects the difference to occur (otherwise it might be averaged out). Leaving any of these empty turns off the statistical comparison. “Test Threshold” can be used to show groups as either hits or not in the board view. Only values above this value will be shown. Although it can be used also when no control group is set, it is probably most useful with a control group, because then a value above 2-3 standard deviations from the control would mean a statistically significant difference, regardless of the score used, and so setting the threshold to 3, e.g., would show all hits found by a certain score. The “Error Display Type” can take one of the values “none”, “all”, “std” or “sem”. The chosen value determines how errors should be displayed in the group view. Respectively, they mean that errors are not displayed at all, that all individual sample points are plotted or that error bars showing standard deviations or standard errors are used. Finally “Default Fly Count” gives the value of number of flies (or, alternatively, the number of biological specimens) in each vial which is used when the “Vial Fly Count” field described in the previous section is left at zero.

Board View.

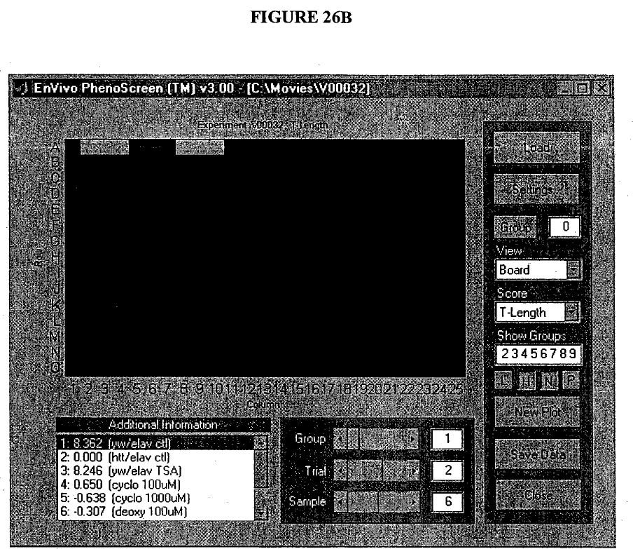

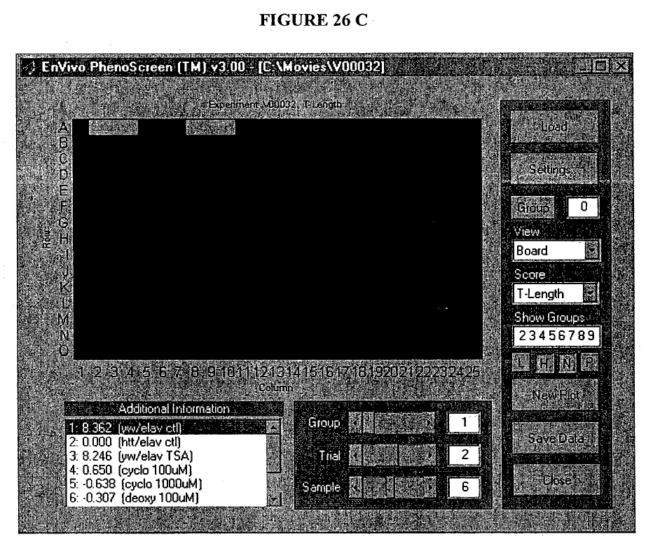

FIGS. 26A-26C are exemplary board views. They each reflect the grouping and the settings made. For example, FIGS. 26A-26C show the same data but with different settings of “Control Group” and “Test Threshold”. Note that all vials within the same group will show the same value since they are used together. In the additional information box the number, score value and name of each group is shown. In the second case, group number 2 has been set as control and what we see now is instead deviation from that group in terms of number of standard deviations. Note that groups 1 and 3 have high values, which is to be expected, while group 2 has a value of zero because it is the control. In the third case, the “Test Threshold” value has been set to 3 to more easily pick out significant hits and groups 1 and 3 are displayed as hits.

Bars View.

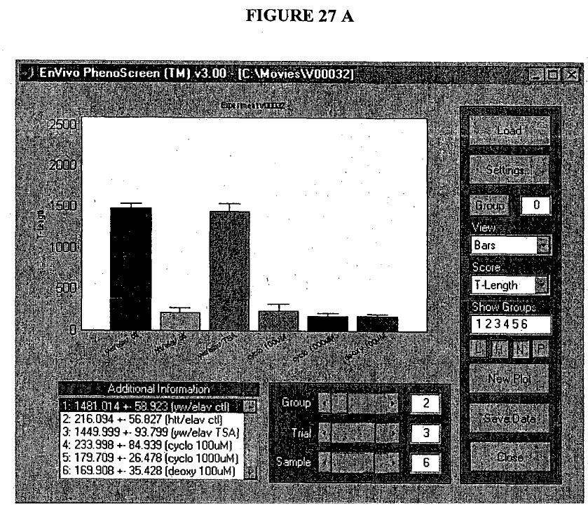

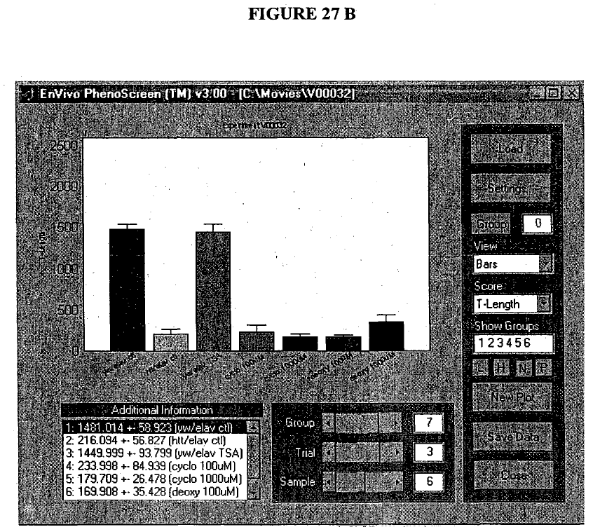

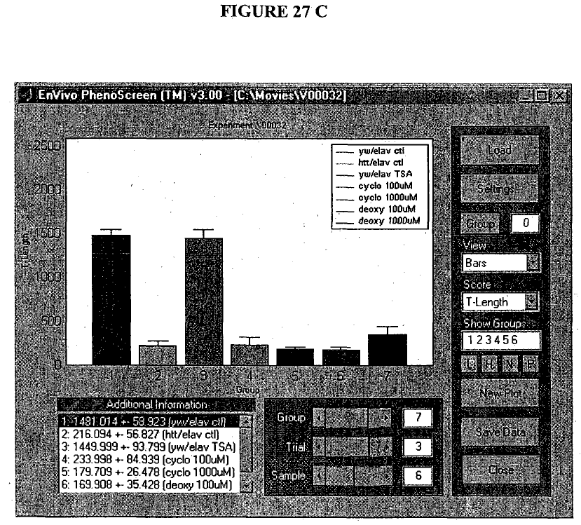

FIGS. 27A-27C are exemplary bar views. This view is very useful for comparing results between groups in a more detailed way than with the board view. For this view, as well as for the group view, the “Show Groups” box and the four one-letter buttons will have an effect. The numbers of the groups desired may be entered to show simultaneously in the “Show Groups” box separated by spaces and press return. That will bring up the bars for those groups in the window with the corresponding group colors, followed by a black bar indicating the active group. The active group is selected using the group slider bar below the plot. It can also be turned off by pressing the “H” (Hide) button, as in FIG. 27A. A user may set some “background” bars consisting of the positive and negative controls using “Show Groups” and then go through and compare the rest using the group slider. The trial slider may be used to flip between trials.

When the “Error Display Type” is set to “std”, standard deviation will be used for error bars and info box, and the title will include the text “StDev” to indicate this. For all other settings, standard error of the mean is used. The “N” (Names) button is used to toggle between showing group numbers or names below the plot. It is on in FIGS. 27A and 27B, but turned off in FIG. 27C. When it is off, an alternative is to use the “L” (Legend) button instead, as in the right figure, to show a legend in the plot. Pressing it repeatedly will move the legend to a chosen position or turn it off completely. When the “P” (Pool) button is in the on position, average values and errors are calculated over all trials, i.e. the same average will be shown as in the board view. Clicking in the plot will take you to the group view, keeping the same active group.

Group View.

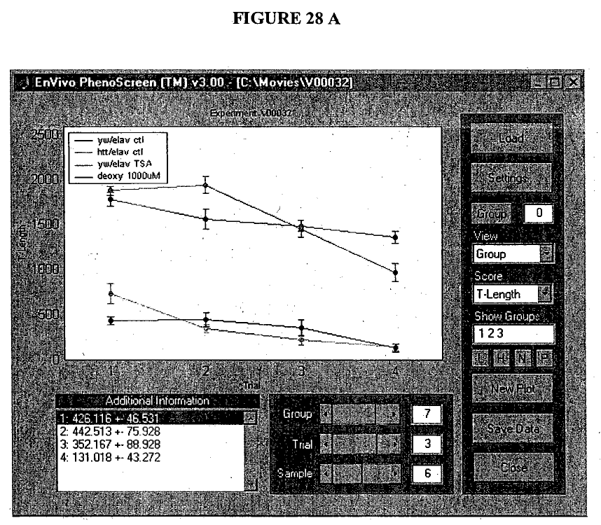

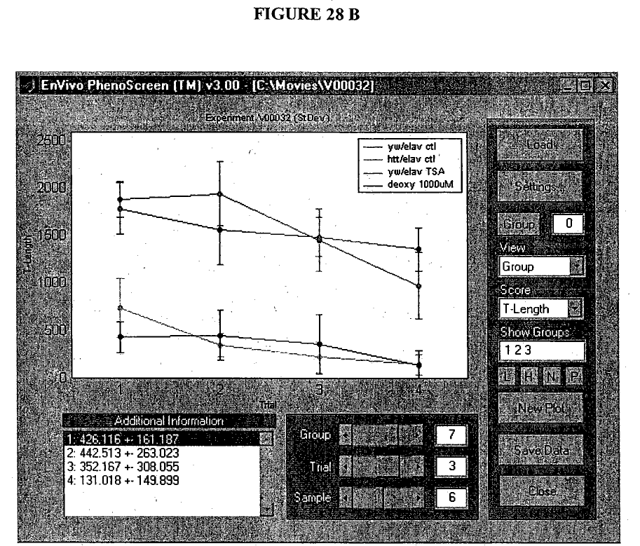

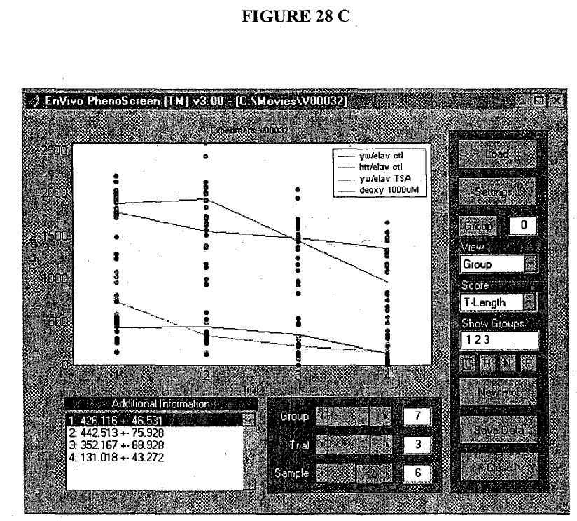

FIGS. 28A-28C show exemplary group views. The “H” and “L” buttons are active also in the group view, and work in exactly in the same way as in the bars view. The same things are true here about the “Error Display Type”, except that also the values “none” and “all” work. In the plots in FIGS. 28A-28C, “sem”, “std” and “all” are used to display the errors. Note also that in the plot shown in FIG. 28A, the legend has been positioned differently. Clicking in the plot takes the user to the trial that was clicked on for the active group in the trial view.

Trial View.

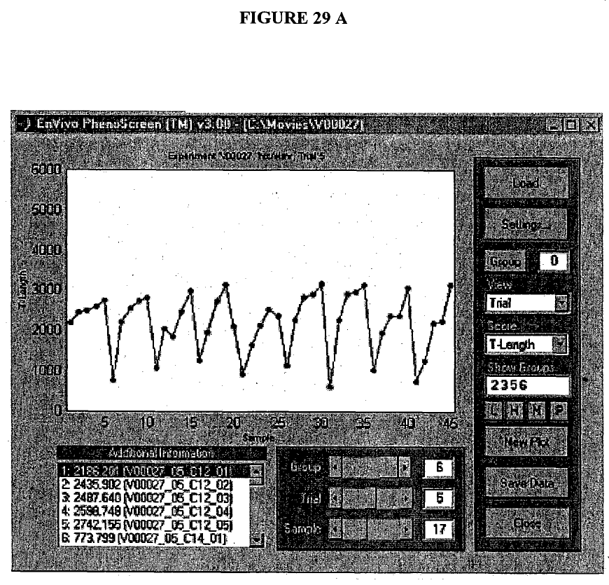

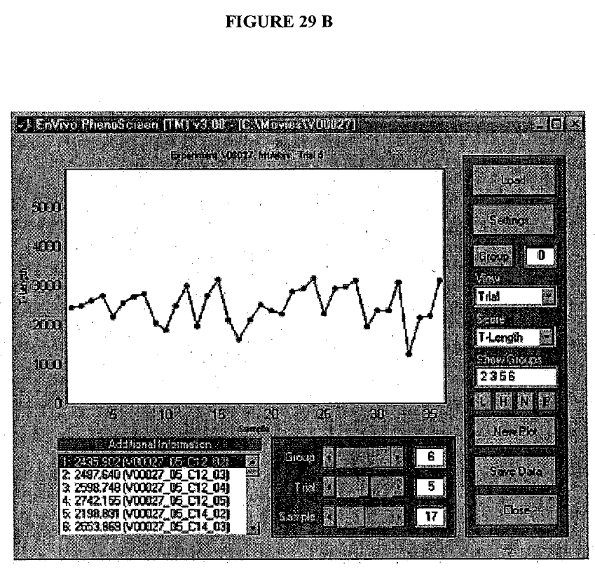

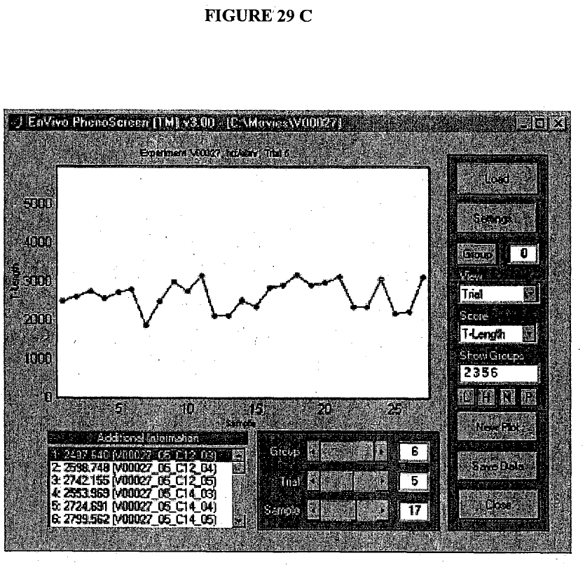

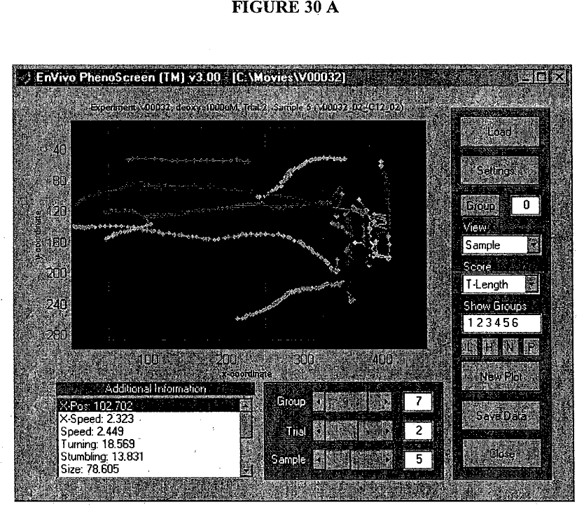

FIGS. 29A-29C show exemplary trial views. All repeats from the vials of a group are shown. (The term sample for all values in a group is used instead of repeats to avoid confusion, since all samples of a group is composed of repeats from multiple vials.) In FIG. 29A, one can see how the first repeat clearly deviates from the others for the V00027 experiment. (Every fifth sample is the first repeat for a vial.) The actual movie names are shown in the info box. Using the “Exclude Repeats” field in the settings dialog we can remove all first repeats, which have been done in FIG. 29B. In FIG. 29C also the second repeats have been removed, which can be seen from the movie names in the information box. Clicking on a data point takes the user to that movie.

Sample View.

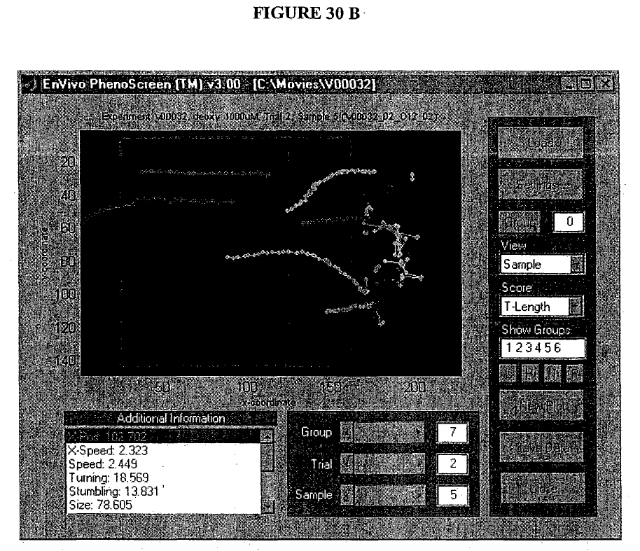

FIGS. 30A and 30B show exemplary sample views. In the sample view, four features are provided. First, to play the movie, one clicks in the frame. Second, the two lines used for high (FIG. 29B) and low (FIG. 29C) cross scores are shown in gray. Third, the frame rectangle is shown with green dashed lines. Last, when playing the movie, during the period within the frames defined by “Frame Subset”, the green rectangle changes to red to indicate that that portion of data is being used. This is demonstrated in FIG. 30B.

Other.

When pressing the “Close” button or when rescoring has to be performed, the current state of the program is saved in the configuration (config) file so that work can be picked up again from where it was left when last exiting.

Description of an Exemplary Configuration File Format.

Below are exemplary individual entries for an assay configuration file:

Configuration: The name of this configuration. For files in the configuration directory this may be the same as the file name without the .cfg extension. For configuration files inside the individual experiment directories this will be the name of the configuration that was used when the experiment was started.

Exp Name: The name of the experiment. For files in the configuration directory this value will be empty. It is filled out when the experiment is first started and the configuration file is copied to the experiment directory.

Exp Comment: The comment of the experiment. For files in the configuration directory this value will be empty. Otherwise it is filled out each time a new trial of the experiment is started.

VISA String: This string is normally “ASRL1::Instr” meaning that COM1 is used for communication with the machine. Unless the machine is connected to another COM port, it should never have to be changed.

Lift Z: The position in 1/100 millimeters from the machine Z reference where the gripper will grab the vial.

Drop Z: The position in 1/100 millimeters from the machine Z reference where the gripper will drop the vial.

Camera Z: The position in 1/100 millimeters from the machine Z reference that the gripper will move to when capturing a movie of the vial.

Movement Z: The position in 1/100 millimeters from the machine Z reference that the gripper will move to before moving from one board position to another.

Origin X, Origin Y: The positions in 1/100 millimeters from the machine X and Y references that the center of the top right board position is located.

Delta X, Delta Y: The distances in 1/100 millimeters between adjacent board positions in the X and Y directions.

Ref Speed X, Ref Speed Y, Ref Speed Z: The speeds in steps/seconds with which the X, Y and Z-axes move to the reference position.

Speed X, Speed Y, Speed Z: The speeds in steps/seconds with which the X, Y and Z-axes move normally.

Nr Repeats: The number of times each vial should be dropped and filmed. NOTE: Zero is a special value, denoting that vials should directly picked up and filmed without being dropped first.

Repeat Delay: The number of milliseconds the program should wait between repeats.

Movie ROI Left, Movie ROI Top, Movie ROI Width, Movie ROI Height: Left and top pixel coordinates, width and height in pixels of movie region of interest (ROI). The ROI is the part of the full camera picture that will be captured.

Nr Frames: The total number of frames that will be captured for each movie.

Skipcount: The number of frames to skip between captured frames. Used to adjust the framerate of the movie capture. A value of zero means that the framerate will be equal to [Max Framerate]. A higher number means the framerate will be equal to [Max Framerate]/([Skipcount]+1).

Capture Delay: The number of milliseconds the program will wait between the arrival of the vial at the camera position and the movie capture.

Storage Path: The directory path of the stored experiment data.

Max Framerate: The maximum framerate of the framegrabber. This value should never be changed unless the framegrabber is exchanged.

Threshold: The thresholding level of the motion tracking software.

Min Area: The minimum blob area that will be detected as a fly by the motion tracking software.

Max Area: The maximum blob area that will be detected as a fly by the motion tracking software.

Prediction Factor: Can assume a value between 0 and 1. The extent to which the motion tracking software will attempt to predict the position of a fly in one frame from its position in the previous frames.

Search Distance: The maximum distance at which the motion tracking software tries to find a fly in the next frame from its predicted position in that frame.

Merge Distance: The maximum distance at which the motion tracking software tries to detect merged blobs.

Split Distance: The maximum distance at which the motion tracking software tries to split up blobs.

Speed Weight: The weight of the speed of the fly (or other specimen) used by the motion tracking software when matching blobs.

Rotate: One or zero depending on whether the compressed movies were also rotated. Should be zero.

Downscale: Pre-compression downscale factor. The value of two means compressed image is half size.

Row A-O: The board setup. All entries should be zero. Updated when a new experiment is created.

Pixels Per mm X: For future conversions to real-world coordinates.

Pixels Per mm Y: For future conversions to real-world coordinates.

Origin mm X: For future conversions to real-world coordinates.

Origin mm Y: For future conversions to real-world coordinates.

Min Elongation: The minimum ratio between length and width for detected flies.

Max Elongation: The maximum ratio between length and width for detected flies.

Control Group: The control group used for statistical comparisons in the analysis program.

Default Fly Count: The default number of flies (or other specimen) in the vials used when no number is explicitly given.

Error Display Type: Takes one of the values “none”, “all”, “sem” or “std”. Selects how to view errors in the group view of the analysis program.

Exclude Repeats: Space-separated array of the repeat numbers that will be excluded from viewing and scoring.

Exclude Trials: Space-separated array of the trial numbers that will be excluded from viewing and scoring.

Fly Count Row A-O: The individual fly (or other specimen) count for each vial position. Each vial has a width of three characters. Zero values mean that the default fly count should be used instead. Used to compensate for different number of flies between vials.

Frame Rectangle: Space-separated array of four values giving x, y, width and height of a rectangle. Data values outside of this rectangle will be disregarded. Negative values of width and height can be used to denote distance from right and bottom edges. All zeros means that the whole frame should be used.

Frame Subset: Space-separated array of two values giving first and last frame of a frame range to be used. Data values from frames before the first frame value or after the last frame value will be disregarded. A negative value of the last frame value can be used to denote the number of frames from the end of the movie. Two zeros means that the all frames should be used.

Group Name 1, 2, . . . : A number of string entries corresponding to the total number of groups as set by the grouping entries. Contains the names for the groups. Note that the numbers do NOT correspond to the actual group numbers, but rather to the position of the group in a list with all groups.

Grouping Row A-O: The group number for each vial. Each vial has a width of three characters. A value of zero for a position with a vial according to the row entries denotes that the vial is in the dummy group and not used.

Last Group: All entries starting in “Last” are used to save information about the state the analysis software was in when last exiting. The value of the group slider when last exiting.

Last Sample: The value of the sample slider when last exiting

Last Score: String entry with the name of the active score when last exiting.

Last Show Groups: Space-separated array with the values of the “Show Groups” box when last exiting.

Last Trial: The value of the trial slider when last exiting.

Last View: String entry with the name of the active view when last exiting.

Last Legend: The state of the legend button when last exiting. A value of 1-4 means counterclockwise position from top right corner. A value of zero means that the legend was turned off.

Last Hide: The state of the hide button when last exiting. Zero or one.

Last Names: The state of the names button when last exiting. Zero or one.

Last Pool: The state of the pool button when last exiting. Zero or one.

Min Nr of Trajectories: Used for scoring. Data from movies with less than this number of trajectories will be disregarded.

Min Trajectory Length: Used for scoring. Data from trajectories with less than this number of points will be disregarded.

Test Trials: Space-separated array with the trial numbers used for the statistical comparisons.

Cross Lines: Used for scoring. Space-separated array of two values giving high and low x-coordinates of the cross scores.

Test Threshold: All values above this one will be shown as hits in the board view of the analysis program. A value of zero means that this functionality is turned off.

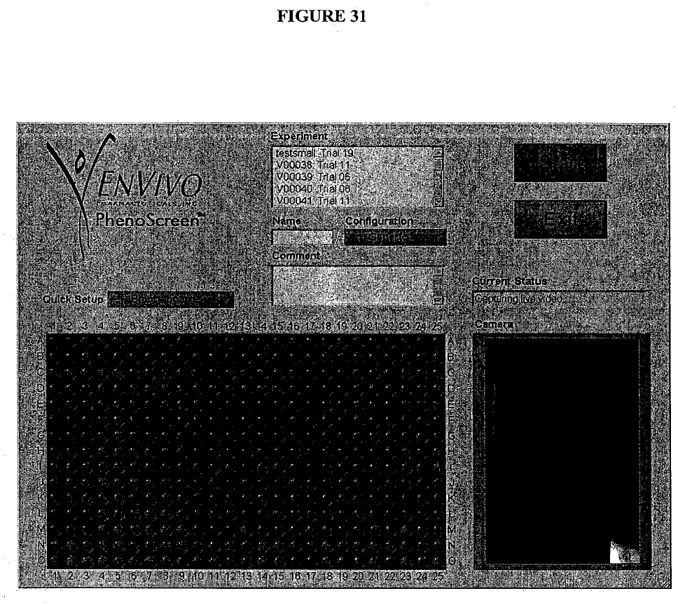

FIG. 31 shows an exemplary screen shot of automation control software. The experiment field includes on-going experiment ID information. The Name field allows one to add a new experiment and ID number. Configuration comprises a pull-down tab to select preset configurations of the machine, including speed of motion, video length, number of repeat video, etc.

The Comments field allows the user to list details or special comments about the experiment or trial. The Quick Setup button allows the user to choose a pre-selected board layout.

The description herein provides new methodology for screening for agents with a desired biological activity. The embodiments are particularly useful for high throughput screening for agents with anti-neurodegenerative activity. The embodiments also provide new and efficient methodology for the quantitative description and/or characterization of one or more traits (e.g., behavior or locomotor activity) associated with an animal disease model. The invention also provides other methods and assays useful for identification of agents with therapeutic activity.

Although the methods of the invention can be applied using a variety of animal populations, as described below, they find particular application when practiced using populations of flies, e.g., Drosophila melanogaster. For convenience, but not for limitation, the description below will generally describe the invention as used when the test biological specimen (e.g., animal) populations is flies.

In one embodiment, the invention provides methods for screening for the effects of a test agent on a population of animals which entail providing a population of animals, administering at least one test agent to the population, creating a digitized movie showing movement of animals in the population, determining one or more traits of the population, and correlating the traits of the population with the effect of the test agent(s) administered to the population. In another embodiment, the invention provides methods for screening for the effects of a test agent on a population of animals which entail providing a plurality of populations of animals, administering at least one test agent to each of the populations, creating image information concerning animals in each population, determining at least two traits of each population and, for each population, correlating the traits of the population with the effect of the test agent(s) administered to the population. In this context, the plurality of populations (e.g., a plurality of samples) is at least 3 populations, and often more than 3, e.g., at least about 10 populations, at least about 20 populations, at least about 100 populations, or at least about 200 populations. In some embodiments of the invention, a large number of test populations are efficiently analyzed, for example, at least about 10 populations, at least about 20 populations, at least about 100 populations, at least about 200 populations, at least about 300 populations, at least about 400 populations or more can be tested in a single day.

Thus, for example methods of the invention are used to screen for biologically active agents in the following manner: Two stocks of Drosophila melanogaster are obtained; a parental stock and a transgenic stock that differs from the parent by virtue of comprising and expressing a transgene that causes a disease phenotype in the flies. An exemplary transgenic fly is a fly that exhibits neurodegeneration as a result of transgene expression.

In one aspect of the invention as encompassed in this illustrative embodiment, a number of traits exhibited by the parental stock and the transgenic stock are measured, and the traits of the two stocks are compared to identify particular traits that distinguish the two stocks. The measured traits usually include movement traits, behavioral traits, and/or morphological traits. In one aspect, the traits are measured by detecting and serially analyzing the movement of a population of flies in containers, e.g., vials. Movement of the flies is monitored by a recording instrument, such as a CCD-video camera, the resultant images are digitized, analyzed using processor-assisted algorithms as described herein, and the analysis data is stored in a computer-accessible manner. For example, in measuring traits related to fly movement, the trajectory of each animal may be monitored by calculation of one or more variables (e.g., speed, vertical only speed, vertical distance, turning frequency, frequency of small movements, etc.) for the animal. Values of such a variable are then averaged for population of animals in the vial and a global value is obtained describing the trait for each population (e.g., parental stock flies and transgenic flies). Global values for each trait are compared and a subset of traits that differs significantly between the populations is identified. The subset of traits and the values of the traits for a particular population (e.g., the parental fly stock) is referred to as a “phenoprint” of that population. Thus, the traits in which a test population of biological specimens differs from a population of control biological specimens is referred to as the “phenoprint” of the test population. Similarly, the traits in which a parental fly stock differs from a transgenic fly stock is the “phenoprint” of the transgenic stock. The phenoprint for a population is a useful tool in the identification of therapeutic agents. For example, an agent that affects various traits of the transgenic fly population with a neurodegenerative phenotype in a fashion that effectively eliminates the phenoprint (e.g., makes the phenoprofile (“phenoprofile” is defined hereinbelow) of the diseased population more similar to the phenoprofile of a control population) of the diseased population is likely to have biological activity protective against the effects of neurodegeneration.

In another aspect of the invention as encompassed in this illustrative embodiment, an automated system is used for high throughput screening of agents with biological activity. In one embodiment, for use in such a system, populations of transgenic flies, e.g., 2-50 flies, are contained in optically transparent vials containing support medium. A different test agent is administered to the flies in each vial, and the automated system is used to determine the traits for each population. Either a single trait may be determined or a number of traits determined to thus generate a phenoprint for the sample population. As above, the traits can be measured by detecting and serially analyzing the movement of a population of flies in containers, e.g., vials. Movement of the flies is monitored by a recording instrument, such as a CCD-video camera, the resultant images are digitized. Movement, behavioral and morphological traits are determined by analysis of the images using processor-assisted algorithms, and the analysis data is stored in a computer-accessible manner as described hereinabove. By comparing a trait or group of traits (e.g., phenoprint) of populations treated with different test agents with each other and/or with reference populations (such as parental wild-type flies) the ability of large numbers of test agents to affect neurodegeneration can be rapidly assessed. For example, the ability of an agent to change at least some traits of a transgenic population with a neurodegenerative phenotype to the traits characteristic of the parental flies is indicative of a desirable biological activity. Thus the methods of the present invention may be used to identify a candidate agent which is useful for modifying a single trait of a population, or alternatively, multiple traits. The high throughput assay system of the invention allows for large scale testing of and/or screening for agents. The analysis of multiple traits (e.g., a phenoprofile), including specific traits described herein, allows the effects of test agents to be determined with much greater precision and sensitivity than other methods.

A wide variety of other embodiments will now be described.

A test population is a population (i.e., sample) of test biological specimens that has come in contact with a test agent. In one aspect of the invention, the effect of a test agent on a test population is determined. More often, the effect of a number of different test agents on a number of different test populations is determined. In the latter case, the test specimens in each of the different test populations is genetically similar or the same (e.g., all of a particular fly strain, all comprising the same transgene, etc., and optionally all male or all female). Thus, the fact that the test agent varies between test populations while the test specimens are constant allows the effect of various test agents to be compared. The size of the population can vary, but for flies it is usually between about 2 and 50 flies (inclusive), for example, between about 5 and about 30 flies, or between about 10 and about 30 flies. Usually the test population is confined in a sample container, such as a vial. Usually the container is optically transparent so that the traits of the population can be recorded.

The effect of the test agent on a test population can be determined by measuring one or more traits exhibited by the test population. Examples of traits that can be measured in the practice of the invention are described in some detail below. Briefly, however, exemplary traits include movement traits (e.g., path length, stumbling, turning, and/or speed), behavioral traits (e.g., appetite, mating behavior, and/or life span), and morphological traits (e.g., shape, size, or location in the animal of a cell, organ or appendage, or size, shape or growth rate of the animal, or the change of any such parameters over time). As is discussed below, movement is of particular interest. In one example, using the automated motion tracking apparatus described herein, movement and behavior traits (particularly behavior trait(s) involving locomotor activity) of populations of flies are assessed over a short period of time (e.g., 1-20 seconds, more often 4 to 10 seconds) after a brief stimulus.

A description (e.g., a quantitative description) of one or more of the measured traits together defines a phenoprofile of the test population. A hypothetical example of a phenoprofile is provided in Table 1, infra. The phenoprofile of a population treated with a specific test agent is referred to as the “agent phenoprofile”.

Another type of phenoprofile is a “reference phenoprofile,” which is a quantitative description of the traits exhibited by a reference population. A reference population may be any of several different populations of biological specimens, and in some methods of the invention, traits of a test population of specimens are compared to traits of a reference population of specimens, or stated somewhat differently, an agent phenoprofile is compared to a reference phenoprofile. Animals used as the reference population in any given assay will generally depend on the test population and/or on the particular method and/or assay performed. For example, when a method involves the use of transgenic flies which express a particular transgene that results in specific behavior trait(s), a reference population may be non-transgenic flies with the same genetic background as the transgenic flies (except for the particular transgene that results in the behavior phenotype). As another example, when a method analyzes a population of flies treated with a test agent, the reference population may be a population of the same flies not treated with the test agent or the reference population may be a population of flies treated with a specified agent, for example an agent that has a known effect on the animals. As another example, when a method involves the use of flies with a genetic alteration which results in a change in level of expression of an endogenous polypeptide (e.g., an alteration which produces a gain of function or a loss of function result), a reference population may be flies without the mutation. In some instances, a reference population may consist of a population of specimens with a different transgene than that of the test population so that a phenotype due to expression of a transgene in a test population can be compared to a phenotype due to the expression of a different transgene in the reference population.

In some embodiments, more than one reference population of specimens is used. For example, when analyzing the effect of a test agent on a test population, the phenoprofile that results from exposure to the agent (the agent phenoprofile) may be compared to a reference phenoprofile of the same population of specimens not treated with a test agent and to a reference phenoprofile of wild-type specimens. It will be apparent that the test and reference populations in any assay are the same species.

The particular traits exhibited by (and thus the particular phenoprofile of) the test and/or reference population(s) is influenced by the genotype of the animal, the properties of any test agent to which the animal is exposed, the age of the animal and other factors. In this context, the term “genotype” is defined broadly and includes, for example, a variety of gene expression events such as the expression of a mutated gene, the failure of expression of a normally expressed gene and/or the expression of a transgene.

Biological specimens, useful in the present invention are preferably animals, and more preferably are generally members of the class insecta, e.g., dipterans and lepidopterans, although in principle other animals, including other invertebrates, e.g., nematodes such as C. elegans, and vertebrates, e.g., zebrafish and mice, may be used in the methods. Of particular use in many embodiments are flies. Examples of such flies include members of the family Drosophilidae, including Drosophila melanogaster. In certain embodiments, the flies are transgenic flies, e.g., transgenic Drosophila melanogaster. A transgenic animal is an animal comprising heterologous DNA (e.g., from a different species) incorporated into its chromosomes. In other embodiments, the animals contain a genetic alteration which results in a change in level of expression of an endogenous polypeptide (e.g., an alteration which produces a gain of function or a loss of function result). The term animal or transgenic animal can refer to animals at any stage of development, e.g. adult, fertilized eggs, embryos, larva, etc.

In particular embodiments, test specimens used in methods of the invention exhibit one or more traits that is indicative of and/or characterizes a neurodegenerative condition in the specimen (e.g., including impaired motor skills, impaired cognition, neuronal cell death, etc.). In some cases, test specimens are flies which exhibit phenotypes which characterize adult onset neurodegenerative disorders, e.g., following the initial hours of adult life, the flies exhibit a neurodegeneration phenotype, including, but not limited to: progressive loss of neuromuscular control, e.g. of the wings; progressive degeneration of general coordination; progressive degenerative of locomotion; and progressive degeneration of appetite. Some flies may also be further characterized in that death occurs prematurely compared to wild-type flies, for example, at 4 to 6 days of adult life. Useful test animals include animal models for adult onset neurodegenerative disorders, such as: Parkinson's Disease, Alzheimer's Disease, Huntington's Disease, spinocerebellar ataxia (SCA), and the like. In addition, the methods of the present invention may be used to assess, and derive therapies for other neurodegenerative diseases including, but not limited to age-related memory impairment, agyrophilic grain dementia, Parkinsonism-dementia complex of Guam, auto-immune conditions (eg Guillain-Barre syndrome, Lupus), Biswanger's disease, brain and spinal tumors (including neurofibromatosis), cerebral amyloid angiopathies (Journal of Alzheimer's Disease vol 3, 65-73 (2001)), cerebral palsy, chronic fatigue syndrome, corticobasal degeneration, conditions due to developmental dysfunction of the CNS parenchyma, conditions due to developmental dysfunction of the cerebrovasculature, dementia-multi infarct, dementia-subcortical, dementia with Lewy bodies, dementia of human immunodeficiency virus (HIV), dementia lacking distinct histology, Dementia Pugilistica, diffues neurofibrillary tangles with calcification, diseases of the eye, ear and vestibular systems involving neurodegeneration (including macular degeneration and glaucoma), Down's syndrome, dyskinesias (Paroxysmal), dystonias, essential tremor, Fahr's syndrome, fronto-temporal dementia and Parkinsonism linked to chromosome. 17 (FTDP-17), frontotemporal lobar degeneration, frontal lobe dementia, hepatic encephalopathy, hereditary spastic paraplegia, hydrocephalus, pseudotumor cerebri and other conditions involving CSF dysfunction, Gaucher's disease, Hallervorden-Spatz disease, Korsakoff's syndrome, mild cognitive impairment, monomelic amyotrophy, motor neuron diseases, multiple system atrophy, multiple sclerosis and other demyelinating conditions (eg leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or virus infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyngeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, and Wilson disease.

In some embodiments, biological specimens for use in methods of the invention are transgenic insects (or other transgenic animals) that harbor a stably integrated transgene that is expressed in a manner sufficient to result in a phenotype different from that of wild-type animals, e.g., a neurodegenerative phenotype. The term “transgene” is used herein to describe genetic material which has been or is about to be artificially inserted into the genome of a cell. In some instances, the transgene must be expressed in a specific manner spatially and/or temporally in the animal to result in the desired phenotype. For example, with regard to a neurodegenerative phenotype, spatial expression of a particular transgene may be limited to neuronal cells. In other instances, specific spatial and/or temporal expression of a transgene is not required to result in the desired phenotype, including a neurodegenerative phenotype.

Examples of transgenes used in insects, such as flies, include, but are not limited to, mammalian transgenes, human transgenes, genes found to be associated with a human disease (e.g., CNS or neurodegenerative disease) and genes that encode proteins associated directly or indirectly with a human disease. For example, introduction of human disease genes with dominant gain-of-function mutations into Drosophila has generated fly models for a number of neurodegenerative diseases. See, for example, Chan et al. (2000); Feany et al. (2000); Fernandez-Funez et al. (2000); Fortini et al. (2000); Jackson et al. (1998); Kazemi-Esfarjani et al. (2000); Warrick et al. (1998); Wittmann et al. (2001) Science 293:711-4.

Examples of genes associated with human neurodegenerative diseases include those identified as having an expanded trinucleotide sequence as compared to the wild-type gene and thus, encode for a polypeptide with an expanded polyglutamine tract as compared to the wild-type polypeptide. Examples of diseases associated with polyglutamine repeats include Huntington's Disease, spinocerebellar ataxia type 1 (SCA1), SCA2, SCA3, SCA6, SCA7, SCA17, spinobulbar muscular atrophy (SBMA) and dentatorubropallidolusyan atrophy (DRPLA) (Cummings et al. (2000) Human Mol. Genet. 9:909-916; Fischbeck (2001) Brain Res. Bull. 56:161-163; Nakamura et al. (2001) Hum. Mol. Genet. 10:1441-1448). For example, expression of the mutated human ataxin-1 in transgenic flies (the polypeptide encoded by the gene associated with SCA1) is accompanied by adult-onset degeneration of neurons, with nuclear inclusions that are immunologically positive for the mutated protein, ubiquitin, Hsp70 and proteosome components (Fernandez-Funez et al., 2000). In addition, in flies which express the SCA1 or SCA3 disease genes, the disease is modified by overexpression of chaperones (Fernandez-Funez et al., 2000; Warrick et al., 1999). Transgenic flies that express exon-1 of huntingtin, a polypeptide encoded by the gene associated with Huntington's Disease and which contains an expanded polyglutamine repeat, demonstrate a progressive neurodegeneration where the time of onset and severity are linked to the length of the polyglutamine repeat (Marsh et al., 2000).

Transgenic Drosophila with neuronal expression of human mutated alpha-synuclein, a polypeptide encoded by a gene associated with Parkinson's disease, demonstrate age-dependent, progressive degeneration of dopamine-containing cells and the presence of Lewy bodies (Feany et al., 2000). These transgenic flies expressing mutated human alpha-synuclein have impaired motor performance (Feany et al. (2002)) and this disease in flies is modified by overexpression of chaperones (Auluck et al. (2002) Science 295:865-868). Transgenic Drosophila expressing tau protein show neurodegeneration (Wittmann et al. (2001) Science 293:711-4).

As noted, the transgenic flies used in the invention generally exhibit at least one measurable behavior and/or morphological phenotype (trait) associated with the expression of the transgene. The phenotype of the transgenic fly may or may not be similar to the behavior and/or morphological phenotype associated with the expression of the transgene, or the gene from which the transgene was derived, in another type of animal, such as a vertebrate.

Transgenic animals for use in the invention can be prepared using any convenient protocol that provides for stable integration of the transgene into the animal genome in a manner sufficient to provide for the requisite expression of the transgene. Methods for preparing transgenic insects, including the use of mobile elements such as PiggyBAC, MINOS, hermes, hobo and mariner, are described in the art. See, for example, Horn et al. (2000) Dev. Genes Evol. 210:630-637; Handler et al. (1999) Insect Mol. Biol. 8:449-457; Lobo et al. (1999) Mol. Gen. Genet. 261:803-810; U.S. Pat. Nos. 6,051,430, 6,218,185, 6,225,121. Methods of random integration of transgenes into the genome of a target Drosophila melanogaster cell(s) are disclosed in U.S. Pat. No. 4,670,388, the disclosure of which is herein incorporated by reference. Methods for preparing transgenic flies, including the use of the P element, are described in the art. See, for example, Brand et al. (1993); Phelps et al. (1998) Methods 14:367-379; Spradling et al. (1982) Science 218:341-347; Spradling (1986) P ELEMENT MEDIATED TRANSFORMATION IN DROSOPHILA: A PRACTICAL APPROACH (ed. D. D. Roberts, IRL Press, Oxford) pp 175-179.

Generally, the transgene is stably integrated into the genome of the animal under the control of a promoter that provides for expression of the transgene. In some cases, the transgene is stably integrated into the genome of the animal in a manner such that its expression is controlled spatially to a desired cell type and/or temporally to a particular developmental stage. In other cases, although transgene expression is required, spatial and/or temporal control of the expression is not necessary for the generation of a phenotype associated with the transgene expression. The transgene may be under the control of any convenient promoter that provides for requisite spatial and temporal expression pattern, if necessary, and the promoter may be endogenous or exogenous. To obtain the desired targeted expression of the randomly integrated transgene, integration of particular promoter upstream of the transgene (e.g., an exogenous promoter), as a single unit in the element or vector may be employed.

When an endogenous promoter is used, a suitable promoter is located in the genome of the animal. The transgene may then be integrated into the fly genome in a manner that provides for direct or indirect expression activation by the promoter, i.e. in a manner that provides for either cis or trans activation of gene expression by the promoter. In other words, expression of the transgene may be mediated directly by the promoter, or through one or more transactivating agents. Where the transgene is under, direct control of the promoter, i.e. the promoter regulates expression of the transgene in a cis fashion, the transgene is stably integrated into the genome of the fly at a site sufficiently proximal to the promoter and, if necessary, in frame with the promoter such that cis regulation by the promoter occurs.

In other embodiments where expression of the transgene is indirectly mediated by the endogenous promoter, the promoter controls expression of the transgene through one or more transactivating agents, usually one transactivating agent, i.e. an agent whose expression is directly controlled by the promoter and which binds to the region of the transgene in a manner sufficient to turn on expression of the transgene. Any convenient transactivator may be employed. For example, in a transgenic fly which uses the GAL4 transactivator system, a GAL4 encoding sequence is stably integrated into the genome of the animal in a manner such that it is operatively linked to the endogenous promoter that provides for expression in the cells of interest. With the GAL4 targeted expression system, the transgene which results in the desired phenotype is generally stably integrated into a different location of the genome, generally a random location in the genome, where the transgene is operatively linked to an upstream activator sequence, i.e. UAS sequence, to which GAL4 binds and turns on expression of the transgene. Transgenic flies having a GAL4/UAS transactivation system are known to those of skill in the art and are described, for example, in Brand et al. (1993); Phelps et al. (1998); and Fernandez-Funez et al. (2000).

In some embodiments, animals for use in methods of the invention are insects (or other animals) that have a mutation that disrupts one or more of their endogenous genes thereby generating a loss-of-function disease phenotype. In Drosophila, for example, genes which are homologs of a human disease genes can be disrupted to produce flies with a loss of function phenotype. See, for example, Reiter et al. (2001) Genome Res. 11:1114-1125 and The et al. (1997) Science 276:791-794.

A variety of loss-of-function mutations in endogenous fly genes have been identified. Examples of such mutations in genes that produce nervous system disorders include swiss cheese (Kretzschmar et al. (1997) J. Neurosci. 17:7425-7432), spongecake, eggroll (Min et al. (1997) Curr. Biol. 7:885-888), drop dead (Buchanan et al. (1993) Neuron 10:839-850), pirouette (Eberl et al. (1997) Proc. Natl. Acad. Sci. USA 94:14837-14842), and bubblegum (Min et al. (1999) Science 284:1985-1988). The bubblegum mutant provides an example of a direct connection between a fly neurodegeneration mutant and a human disease. Both bubblegum flies and patients with the metabolic disorder adrenoleukodystrophy (ALD) accumulate abnormal amounts of very long chain fatty acids (VLCFAs). The bubblegum mutant flies have a mutation in the VLCFA acyl coenzyme A synthetase gene. This enzyme has reduced activity in patients with ALD. Primary defects in glial cells have been implicated as an important mechanism of neurodegeneration in Drosophila. The drop dead and swiss cheese mutants show glial abnormalities before neurons degenerate. Similarly, primary glial cell defects underlie neurodegeneration in some forms of human hereditary peripheral nerve degeneration, such as Charcot-Marie-Tooth disease (Bennett et al. (2001) Curr. Opin. Neurol. 14:621-627).

Examples of loss-of-function mutations in flies that produce stereotypic paralysis and seizures include easily shocked (eas) and slamdance (sda) (Pavlidis et al. (1994) Cell 79:23-33; Kuebler et al. (2001) J. Neurophysiol. 86:1211-1225). Drosophila is a faithful system to identify factors that suppress seizure susceptibility. For example, anti-epileptic drugs such as Gabapentin, Topiramate and Phenyloin administered orally to flies reduce seizure and mean recovery times following seizure (Reynolds et al. (2002) 43^{rd} Annual Drosophila Genetics Conference).

For use in the invention, animals can be prepared by any protocol that disrupts the expression of a gene or genes. For example, the disruption of genes in Drosophila may be accomplished by using P-element transposons (Rubin et al. (1982) Science 218:348-353), chromosomal aberrations may be generated in Drosophila by subjecting flies to irradiation (Sullivan et al. (2000) Drosophila Protocols (2000) Cold Spring Harbor Laboratory Press, New York, pp. 592-593). Additionally, single-base-pair mutations can be can be generated in fly genes with chemical mutagens such as ethylmethanesulfonate (EMS) or ethylnitrosourea (ENU) (Sullivan et al. (2000)). The ability to identify chemically generated point mutations using a set of single nucleotide polymorphisms which span the Drosophila genome has broadened this approach by facilitating chemical-mutagen suppressor screens of a given loss of function phenotype. See, for example, Lukacsovich et al. (2001) Genetics 157:727-742; Berger et al. (2001) Nat. Genet. 29:475-481.

In some embodiments, animals for use in methods of the invention are wild-type insects (or other animals) that suffer from age-related motor dysfunction and age-related death. As in humans, flies demonstrate poor motor performance in latter weeks of their life (Fernandez et al. (1999) Experimental Gerontology 34:621-631; Le Bourg (1987) Experimental Gerontology 4:359-369). Feeding Drosophila with 4-phenylbutyrate (PBA) can significantly increase lifespan, without diminution of locomotor vigor (Kang et al. (2002) Proc. Natl. Acad. Sci. USA 99:838-843).

In some embodiments, animals for use in methods of the invention are wild-type insects (or other animals) that are subjected to environmental stimuli or treated with a substance that produces a disease-like state. For example, rest behavior in Drosophila is a sleep-like state where the animals choose a preferred location, become immobile for periods at a particular time in the circadian day, and are relatively unresponsive to sensory stimuli (Hendricks et al. (2000) Neuron 25:129-138). Rest is affected by both homeostatic and circadian influences and when rest is prevented, the flies increasingly tend to rest despite stimulation and then exhibit a rest rebound. Drugs which act on a mammalian adenosine receptor alter rest as they do sleep, suggesting conserved neural mechanisms. In other examples, wild-type Drosophila demonstrate behavioral traits that resemble aggression when they are placed in a competitive situation, such as courtship (Chen et al. (2002) Proc. Natl. Acad. Sci. USA 99:5664-5668) and Drosophila are sensitive to a depression-like or stress-like environment [Le Bourg et al. (1999) Experimental Gerontology 34:157-172; Le Bourg et al. (1995) Behavioural Processes 34:175-184).

Animals treated with a substance for use in the invention, for example, include wild-type animals exposed to an addictive substance. Upon exposure to ethanol or other addictive substances, wild-type Drosophila display behaviors that are similar to intoxication and addiction seen in rodents and humans (Bellen (1998) Cell 93:909-912). One example of a fly mutant with enhanced sensitivity to ethanol is cheapdate (Moore et al. (1998) Cell 93:997-1007). Other addictive substances for use in the animals may include, for example, cocaine and nicotine (Bainton et al. (2000) Curr Biol. 10:187-194; Torres et al. (1998) Synapse 29:148-161).

Chemical-induced models of human disease in animals include, for example, those which target dopamine neurons such as 1-methyl-4-phenyl-1,2,3,6-tetrahydropyridine (MPTP) or 6-hydroxydopamine (6-OHDA) (Beal (2001) Nat. Rev. Neurosci. 2:325-334). Other examples of chemicals for the generation of such models include, but are not limited to, cholinergic agonists, carbachol, muscarine, pilocarpine, and acetylcholine (Gorczyca et al. (1991) J. Neurobiol. 22:391-404). Additionally, olfactory sensitivity, shock reactivity, and locomotor behavior in flies can be manipulated with hydroxyurea (de Belle et al. (1994) Science 263:692-695).

A phenoprofile of a test or reference population is determined by measuring traits of the population. The present invention allows simultaneous measurement of multiple traits of a population. Although a single trait may be measured, more often at least 2, 3, 4, 5, 7 or 10 traits are assessed for a population. The traits measured can be solely movement traits, solely morphological traits or a mixture of traits in multiple categories. In some embodiments at least one movement trait and at least one non-movement trait is assessed.

In some embodiments, the animal trait(s) measured comprise physical trait data. As used herein, “physical trait data” refers to, but is not limited to, movement trait data (e.g., animal behaviors related to locomotor activity of the animal), and/or morphological trait data, and/or behavioral trait data. Examples of such “movement traits” include, but are not limited to:

a) total distance (average total distance traveled over a defined period of time);

b) X only distance (average distance traveled in X direction over a defined period of time;

c) Y only distance (average distance traveled in Y direction over a defined period of time);

d) average speed (average total distance moved per time unit);

e) average X-only speed (distance moved in X direction per time unit);

f) average Y-only speed (distance moved in Y direction per time unit);

g) acceleration (the rate of change of velocity with respect to time);

h) turning;

i) stumbling;

j) spatial position of one animal to a particular defined area or point (examples of spatial position traits include (1) average time spent within a zone of interest (e.g., time spent in bottom, center, or top of a container; number of visits to a defined zone within container); (2) average distance between an animal and a point of interest (e.g., the center of a zone); (3) average length of the vector connecting two sample points (e.g., the line distance between two animals or between an animal and a defined point or object); (4) average time the length of the vector connecting the two sample points is less than, greater than, or equal to a user define parameter; and the like);

m) path shape of the moving animal, i.e., a geometrical shape of the path traveled by the animal (examples of path shape traits include the following: (1) angular velocity (average speed of change in direction of movement); (2) turning (angle between the movement vectors of two consecutive sample intervals); (3) frequency of turning (average amount of turning per unit of time); (4) stumbling or meandering (change in direction of movement relative to the distance); and the like. This is different from stumbling as defined above. Turning parameters may include smooth movements in turning (as defined by small degrees rotated) and/or rough movements in turning (as defined by large degrees rotated).

“Movement trait data” as used herein refers to the measurements made of one or more movement traits. Examples of “movement trait data” measurements include, but are not limited to X-pos, X-speed, speed, turning, stumbling, size, T-count, P-count, T-length, Cross150, Cross250, and F-count. Descriptions of these particular measurements are provided below.

X-Pos: The X-Pos score is calculated by concatenating the lists of x-positions for all trajectories and then computing the average of all values in the concatenated list.

X-Speed: The X-Speed score is calculated by first computing the lengths of the x-components of the speed vectors by taking the absolute difference in x-positions for subsequent frames. The resulting lists of x-speeds for all trajectories are then concatenated and the average x-speed for the concatenated list is computed.

Speed: The Speed score is calculated in the same way as the X-Speed score, but instead of only using the length of the x-component of the speed vector, the length of the whole vector is used. That is, [length]=square root of ([x-length]²+[y-length]²).

Turning: The Turning score is calculated in the same way as the Speed score, but instead of using the length of the speed vector, the absolute angle between the current speed vector and the previous one is used, giving a value between 0 and 90 degrees.

Stumbling: The Stumbling score is calculated in the same way as the Speed score, but instead of using the length of the speed vector, the absolute angle between the current speed vector and the direction of body orientation is used, giving a value between 0 and 90 degrees.

Size: The Size score is calculated in the same way as the Speed score, but instead of using the length of the speed vector, the size of the detected fly is used.

T-Count: The T-Count score is the number of trajectories detected in the movie.

P-Count: The P-Count score is the total number of points in the movie (i.e., the number of points in each trajectory, summed over all trajectories in the movie).

T-Length: The T-Length score is the sum of the lengths of all speed vectors in the movie, giving the total length all flies in the movie have walked.

Cross150: The Cross150 score is the number of trajectories that either crossed the line at x=150 in the negative x-direction (from bottom to top of the vial) during the movie, or that were already above that line at the start of the movie. The latter criteria was included to compensate for the fact that flies sometimes don't fall to the bottom of the tube. In other words this score measures the number of detected flies that either managed to hold on to the tube or that managed to climb above the x=150 line within the length of the movie.

Cross250: The Cross250 score is equivalent to the Cross150 score, but uses a line at x=250 instead.

F-Count: The F-Count score counts the number of detected flies in each individual frame, and then takes the maximum of these values over all frames. It thereby measures the maximum number of flies that were simultaneously visible in any single frame during the movie.

The assignment of directions in the X-Y coordinate system is arbitrary. For purposes of this disclosure, “X” refers to the vertical direction (typically along the long axis of the container in which the flies are kept) and “Y” refers to movement in the horizontal direction (e.g., along the surface of the vial).

For each of the various trait parameters described, statistical measures can be determined. See, for example, PRINCIPLES OF BIOSTATISTICS, second edition (2000) Mascello et al., Duxbury Press. Examples of statistics per trait parameter include distribution, mean, variance, standard deviation, standard error, maximum, minimum, frequency, latency to first occurrence, latency to last occurrence, total duration (seconds or %), mean duration (if relevant).

Certain other traits (which may involve animal movement) can be termed “behavioral traits.” Examples of behavioral traits include, but are not limited to, appetite, mating behavior, sleep behavior, grooming, egg-laying, life span, and social behavior traits, for example, courtship and aggression. Social behavior traits may include the relative movement and/or distances between pairs of simultaneously tracked animals. Such social behavior trait parameters can also be calculated for the relative movement of an animal or between animal(s) and zones/points of interest. Accordingly, “behavioral trait data” refers to the measurement of one or more behavioral traits. Examples of such social behavior trait traits include, for example, the following:

a) movement of one animal toward or away from another animal;

b) occurrence of no relative spatial displacement of two animals;

c) occurrence of two animals within a defined distance from each other;

d) occurrence of two animals more than a defined distance away from each other.

In addition to traits based on specimen movement and/or behavior, other traits of the specimens may be determined and used for comparison in the methods of the invention, such as morphological traits. As used herein, “morphological traits” refer to, but are not limited to gross morphology, histological morphology (e.g., cellular morphology), and ultrastructural morphology. Accordingly, “morphological trait data” refers to the measurement of a morphological trait. Morphological traits include, but are not limited to, those where a cell, an organ and/or an appendage of the specimen is of a different shape and/or size and/or in a different position and/or location in the specimen compared to a wild-type specimen or compared to a specimen treated with a drug as opposed to one not so treated. Examples of morphological traits also include those where a cell, an organ and/or an appendage of the specimen is of different color and/or texture compared to that in a wild-type specimen. An example of a morphological trait is the sex of an animal (i.e., morphological differences due to sex of the animal). One morphological trait that can be determined relates to eye morphology. For example, neurodegeneration is readily observed in a Drosophila compound eye, which can be scored without any preparation of the specimens (Fernandez-Funez et al., 2000, Nature 408:101-106; Steffan et. al, 2001, Nature 413:739-743). This organism's eye is composed of a regular trapezoidal arrangement of seven visible rhabdomeres produced by the photoreceptor neurons of each Drosophila ommatidium. Expression of mutant transgenes specifically in the Drosophila eye leads to a progressive loss of rhabdomeres and subsequently a rough-textured eye (Fernandez-Funez et al., 2000; Steffan et. al, 2001). Administration of therapeutic compounds to these organisms slows the photoreceptor degeneration and improves the rough-eye phenotype (Steffan et. al, 2001). In one embodiment, animal growth rate or size is measured. For example Drosophila mutants that lack a highly conserved neurofibromatosis-1 (NF1) homolog are reduced in size, which is a defect that can be rescued by pharmacological manipulations that stimulate signalling through the cAMP-PKA pathway (The et al., 1997, Science 276:791-794; Guo et al., 1997, Science 276:795-798).

Traits exhibited by the populations may vary, for example, with environmental conditions, age of a specimen and/or sex of a specimen. For traits in which such variation occurs, assay and/or apparatus design can be adjusted to control possible variations. Apparatus for use in the invention can be adjusted or modified so as to control environmental conditions (e.g., light, temperature, humidity, etc.) during the assay. The ability to control and/or determine the age of a fly population, for example, is well known in the art. For those traits which have a sex-specific bias or outcome, the system and software used to assess the trait can sort the results based a detectable sex difference in of the specimens. For example, male and female flies differ detectably in body size. Thus, analysis of sex-specific traits need not require separated male and/or female populations. However, sex-specific populations of specimens can be generated by sorting using manual, robotic (automated) and/or genetic methods as known in the art. For example, a marked-Y chromosome carrying the wild-type allele of a mutation that shows a rescuable maternal effect lethal phenotype can be used. See, for example, Dibenedetto et al. (1987) Dev. Bio. 119:242-251.

The present invention makes use of an automated system to provide a quantitative description of traits and determine phenoprofiles. An automated system is a system that includes one or more of the following features or elements: a short cycle time, operates continuously and/or requires little or no manual intervention. For example, such a system would be a motion tracking apparatus and would include a machine apparatus coupled to a robotic system for handling containers of animals (i.e., sample containers), a computer-vision system to measure animal traits and a system to archive the output.

In one embodiment, a large number of test populations are analyzed using the automated system, for example, at least about 10 populations, at least about 20 populations, at least about 100 populations, at least about 200 populations, at least about 300 populations, at least about 400 populations or more can be tested in a single day.

In an aspect, the invention provides a system useful for the practice of the screening and analysis methods described herein. Generally the system includes a sample platform having an array of sample containers suitable for housing animals. For example, the animals can be insects (e.g., flies) or other invertebrates. Generally the system includes a nonvisual detection means (camera) configured to capture a movie of the movement of animals in the container, and a robot configured to move the containers into a position such that the animals in the container can be viewed by the camera, and a processor configured to process the movie captured by the camera. In one embodiment, the robot is configured to remove a container from the platform, position the container in front of the camera, and return the container to the platform. In the practice of the invention with flies, the sample containers (e.g., vials, tubes) contain nutrient medium, for example, including agar support medium, food and/or yeast paste (with or without test agent), and a population of about 2 to about 50, about 5 to about 30, about 10 to about 30, about 10 to about 40, or typically about 10 to about 20, flies. If desired, the files can be reared, stored and assayed (one or more times) in the same sample container.

As discussed above, the term “phenoprofile” refers to a trait or, more usually, a combination of traits exhibited by a population of animals exposed to a test agent (i.e., an agent phenoprofile) or a reference population (i.e., a reference phenoprofile). The traits are described by a quantitative or qualitative value. For illustration, three hypothetical phenoprofiles with arbitrary units are shown in Table 1.

TABLE 1

| | Phenoprofiles | | |
|---|---|---|---|
| | | | Reference |
| Trait measured | Test Population 1 | Test Population 2 | Population |
| x-only speed | 5 | 1 | 6 |
| stumbling | 12 | 25 | 10 |
| path length | 100 | 25 | 100 |
| turning | 45 | 50 | 66 |

Usually, the phenoprofile is defined by measurements of 1, 2, 3, 4, 5, 7 or 10 or more traits. The traits can be solely movement traits, solely behavioral traits, solely morphological traits or a mixture of traits in multiple categories. Preferably, a phenoprofile is comprised of a combination of movement traits and traits from at least one other category. In some embodiments the phenoprofile is determined by measurement of at least 2, 3, 4, often 5, and sometimes 7 movement traits.

In one embodiment, a trait and/or phenoprofile is determined for a specimen population as a whole. In such a case the result for one population can be compared to the result for another population. In another embodiment, a trait and/or phenoprofile is determined for individual animals specimens in a population. For example, when a social behavior trait is evaluated, relationship between individuals of the population is determined and used to generate a phenoprofile. Phenoprofiles can be determined for a large number of test populations as well as for reference populations. In one aspect of the invention, the phenoprofiles of test and/or reference populations are compared with each other.

Since the traits that define phenoprofiles can be stored electronically, comparison of phenoprofiles is conveniently accomplished using computer implemented multivariate analysis. It should be noted that the multivariate analysis can be implemented using any commercially available multivariate analysis package, such as Spotfire DecisionSite, which is available from Spotfire of Somerville, Mass. (SPOTFIRE is a registered trademark). Alternatively, a custom multivariate analysis algorithm can be developed and applied to the recorded traits.

Comparison of phenoprofiles can be carried out to achieve several different goals. In one embodiment, a plurality of agent phenoprofiles are ranked according to their similarity to a reference phenoprofile. Such ranking can be used to screen or rank agent according to their biological effect on the specimens. For example, and not limitation, if the test populations comprise flies exhibiting traits of a neurodegenerative condition, test agents can be screened for the ability to ameliorate the symptoms of the condition by (1) comparing the phenoprofiles of test populations exposed to various test agents with a reference phenoprofile of a healthy (e.g., wild-type) specimens, with test agents that produce phenoprofiles more similar to the reference phenoprofile being ranked higher than test agents that produce phenoprofiles less similar to the reference phenoprofile and/or (2) comparing the phenoprofiles of the test populations with a reference phenoprofile of a test specimen (i.e., exhibiting traits of the neurodegenerative condition), with test agents that produce phenoprofiles less similar to the reference phenoprofile being ranked higher than test agents that produce phenoprofiles more similar to the reference phenoprofile. Thus, in some embodiments, comparison of an agent phenoprofile to a reference phenoprofile is used to select an agent that results in a desired activity, such as ability to produce an agent phenoprofile that is similar to a phenoprofile of a healthy (e.g., wild-type) animal.

In one embodiment, the test animals are transgenic flies expressing a transgene whose expression results, indirectly or directly, in the neurodegenerative condition in the animal. Examples of such transgenes are genes encoding for a polypeptide with an expanded polyglutamine tract as compared to the wild-type polypeptide, such as genes whose expression results in or contributes to Huntington's Disease, spinocerebellar ataxia type 1 (SCA1), SCA2, SCA3, SCA6, SCA7, SCA17, spinobulbar muscular atrophy, dentatorubropallidolusyan atrophy (DRPLA), and other diseases known in the art or to be discovered. In an embodiment, the reference phenoprofile is of a wild-type fly or a fly treated with an agent known to ameliorate the disease condition when administered to mammals with the disease. In one embodiment the reference phenoprofile is of a fly treated with a agent known to reduce the manifestation of at least one trait associated with expression of the transgene.

It will be appreciated that many other types of comparisons are possible depending on the specific aims of the screen. For example, the agent phenoprofiles can be compared with each other or with a reference phenoprofile of an animal treated with an specified agent whose biological activity is known or suspected.

In some instances, methods of the invention are used to determine whether an agent can delay onset of a phenotype of a biological specimen, for example, a phenotype associated with a particular gene expression event, such as expression of a gene associated with a neurodegenerative disease, or alternatively, whether an agent can mitigate or prevent the onset of disease. As used herein, “prevent” means that an animal does not present with a phenoprint of the disease condition within the time during which an animal not exposed to the agent would be expected to develop traits characteristic of the particular disease. As used herein, “mitigate” refers to a decrease in the severity of disease traits, as quantitated using the methods and parameters of the present invention, of at least 10% compared to an animal, equally disposed to develop a particular disease, which has not been exposed to the candidate agent. In such methods, the agent phenoprofile is determined at multiple times during development of the biological specimen. Comparison of the agent phenoprofile and the reference phenoprofile at the various time points is used to determine whether contact with the agent delays onset of the phenotype. In one embodiment, the methods of the present invention may be used to identify a candidate agent which may be useful for the treatment of one or more neurodegenerative diseases including, but not limited to age-related memory impairment, agyrophilic grain dementia, Parkinsonism-dementia complex of Guam, auto-immune conditions (eg Guillain-Barre syndrome, Lupus), Biswanger's disease, brain and spinal tumors (including neurofibromatosis), cerebral amyloid angiopathies (Journal of Alzheimer's Disease vol 3, 65-73 (2001)), cerebral palsy, chronic fatigue syndrome, corticobasal degeneration, conditions due to developmental dysfunction of the CNS parenchyma, conditions due to developmental dysfunction of the cerebrovasculature, dementia-multi infarct, dementia-subcortical, dementia with Lewy bodies, dementia of human immunodeficiency virus (HIV), dementia lacking distinct histology, Dementia Pugilistica, diffues neurofibrillary tangles with calcification, diseases of the eye, ear and vestibular systems involving neurodegeneration (including macular degeneration and glaucoma), Down's syndrome, dyskinesias (Paroxysmal), dystonias, essential tremor, Fahr's syndrome, fronto-temporal dementia and Parkinsonism linked to chromosome 17 (FTDP-17), frontotemporal lobar degeneration, frontal lobe dementia, hepatic encephalopathy, hereditary spastic paraplegia, hydrocephalus, pseudotumor cerebri and other conditions involving CSF dysfunction, Gaucher's disease, Hallervorden-Spatz disease, Korsakoff's syndrome, mild cognitive impairment, monomelic amyotrophy, motor neuron diseases, multiple system atrophy, multiple sclerosis and other demyelinating conditions (eg leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or virus infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyngeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome; Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, and Wilson disease.

It will be appreciated that “comparison” of phenoprofiles does not imply that the compared phenoprofiles were necessarily produced at the same time. For example, a reference phenoprofile can be generated and stored (in electronic form) at one time and agent phenoprofiles generated at different times can be compared to the reference phenoprofile. Conveniently, traits (e.g., fly movement) can be recalled from the recorded movies. Thus, traits (e.g., movement) of each population can be measured multiple times and, if desired, can be conducted many times over the course of the life span (e.g., adult life span) of the flies.

For example, in one aspect, the invention provides a method for determining whether a test agent delays onset of a phenotype in a transgenic fly by providing population of transgenic flies, wherein the population develops a phenotype due to expression of a transgene (e.g., an adult onset disorder, contacting the flies with test agents, and determining an agent phenoprofile for the population in at a plurality of times during the life of the fly). The agent phenoprofile generated at each of the times is compared to a reference phenoprofile generated at corresponding times in a reference population (e.g., transgenic flies not contacted with the test agent), and it is determined whether the test agent delays onset of a phenotype in a population contacted with a test agent compared to the reference population.

In a related aspect, the invention provides a method for identifying a defined set of traits, called a “phenoprint”, that distinguish one population from a second population. This aspect of the invention can best be described by reference to a particular example, i.e., a set of traits that distinguishes a Drosophila population consisting of fly models of neurodegenerative diseases (i.e., flies transgenic for genes or gene fragments associated with Parkinson's disease, Huntington's disease and SCA1, for example) and a Drosophila population consisting of healthy flies (i.e., a wild-type, non-transgenic fly). It is believed that for two such populations (as well as for other combinations of populations) there will be some traits (movement, morphological or behavioral) for which the populations will differ significantly and some traits for which they will not differ. A useful phenoprint consists of traits that do differ, e.g., significantly (e.g., p<0.05). By way of illustration, a phenoprofile for a Drosophila polyglutamine transgenic fly could be, for example, “x-only speed of 5, stumbling of 1000, path length of 98, and turning of 3.” A phenoprint for a particular pair of populations can be determined by comparing traits of each population and identifying or selecting traits that differ most (or significantly) between the two populations.

TABLE 2

| | Reference | Test Population | |
|---|---|---|---|
| | Population | Phenoprofile | Reference |
| | Phenoprofile | (huntington disease | Population |
| Trait measured | (wild-type fly) | transgenic fly) | Phenoprint |
| x-only speed | 6 | 5 | |
| stumbling | 10 | 1000 | 10 |
| path length | 100 | 98 | |
| turning | 66 | 3 | 66 |
| X only distance | 1000 | 998 | |
| average Y-only | 20 | 500 | 20 |
| speed | | | |
| average speed | 20 | 18 | |
| acceleration | 50 | 60 | |

Identification of phenoprints that characterize a particular disease model will be useful, for example, for identifying sensitive and appropriate parameters of motor performance for automated screening for agents that can alter the disease-associated behavior phenotype, in particular, for agents that correct a behavior phenotype toward a wild-type animal behavior phenotype or for agents that delay development of a phenotype associated with a particular disease gene expression event. For example, with reference to Table 2, an exemplary assay could use huntington disease transgenic flies as test animals and screen test agents for the ability to modify the stumbling, turning, and average Y-only speed in a test population to a value close to (or closer to) the reference population phenoprint. Of course, also the variation of the values above has to be considered, and can moreover be used to create an optimal weighted combination of trait values for discrimination purposes. The way of combining them can e.g. be a linear combination or a non-linear one found by means of a neural network or other methods.

A phenoprint determined at a particular time can be compared to a phenoprint determined at a different time and the rate of change in a phenoprint over time, if any, can be determined. Accordingly, the rate of change of a phenoprint for a particular pair of populations can be determined by comparing phenoprints over time of each population.

It will be apparent to the careful reader that a “phenoprint” is a type of “phenoprofile,” and that any comparison, ranking, etc., that can be carried out using phenoprofiles (such as described herein) can be carried out using phenoprints.

As noted above, the agent phenoprofile corresponding to a particular test agent can be used to determine the biological activity of the agent. Alternatively, when the biological activity of an agent is known or suspected, the agent can be used to determine the agent phenoprofile. It will be appreciated that, although the term “test agent” is used to describe the agents, the activity of the agent can be known or unknown.

Agents to be screened can be naturally occurring or synthetic molecules. Agents can be obtained from natural sources, such as, e.g., marine microorganisms, algae, plants, fungi, etc. Agents can include, e.g., pharmaceuticals, therapeutics, environmental, agricultural, or industrial agents, pollutants, cosmeceuticals, drugs, organic compounds, lipids, fatty acids, steroids, glucocorticoids, antibiotics, peptides, proteins, sugars, carbohydrates, chimeric molecules, purines, pyrimidines, derivatives, structural analogs or combinations thereof.

Usually, collections of compounds (known as libraries) are used. Libraries of natural compounds in the form of bacterial, fungal, plant and animal extracts are available or readily produced. Alternatively, agents to be assayed can be from combinatorial libraries of agents, including peptides or small molecules, or from existing repertories of chemical compounds synthesized in industry, e.g., by the chemical, pharmaceutical, environmental, agricultural, marine, drug, and biotechnological industries. Preparation of combinatorial chemical libraries is well known to those of skill in the art. Compounds that can be synthesized for combinatorial libraries include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. Devices for the preparation of combinatorial libraries are commercially available (see, e.g., 357 MPS, 390 MPS, Advanced Chem Tech, Louisville, Ky., Symphony, Rainin, Woburn, Mass., 433A Applied Biosystems, Foster City, Calif., 9050 Plus, Millipore, Bedford, Mass.). Compounds to be screened can also be obtained from governmental or private sources, including, for example, the National Cancer Institute's (NCI) Natural Product Repository, Bethesda, Md.; the NCI Open Synthetic Compound Collection, Bethesda, Md.; NCI's Developmental Therapeutics Program; ComGenex, Princeton, N.J.; Tripos, Inc., St. Louis, Mo.; 3D Pharmaceuticals, Exton, Pa.; and Martek Biosciences, Columbia, Md.

For example, two companies sell libraries of known bioactive or FDA-approved drugs which may be used in methods of the invention. MicroSource Discovery Systems, Inc. (Gaylordsville, Conn.) provides a Gen-PlusTM collection of 960 known bioactive compounds, which contains significant overlap with the National Institute for Neurological Disorders and Stroke (NINDS) compound collection selected for the NINDS screening study. This set permits the simultaneous-evaluation of hundreds of marketed drugs and biochemical standards. Prestwick Chemical (Washington, D.C.) sells a library containing a collection of 640 high-purity chemical compounds the majority of which are off-patent marketed drugs.

Additionally, natural or synthetically produced libraries and compounds are readily modified through conventional chemical, physical and biochemical means, and may be used to produce combinatorial libraries.

Screening may also be directed to known pharmacologically active compounds and analogs thereof. Known pharmacological agents may be subjected to directed or random chemical modifications, such as acylation, coalkylation, esterification, amidification, etc. to produce structural analogs. New potential test agents may also be created using methods such as rational drug design or computer modeling.

As described above, compounds that may be assayed according to the methods of the invention encompass numerous chemical classes. For example, organic molecules, preferably small organic compounds having a molecular weight of more than 50 and less than about 2,500 daltons, are a type of compound for use in the methods of the invention.

One exemplary library for use in methods of the invention includes compounds based on 2,5-diketopiperazine (DKP) scaffold. Generally, compounds of this library are biased toward particular amines, exhibit stability to proteolysis, have a molecular weight range of about 250 to about 450 daltons and have solubilities greater than about 5 mM. Another exemplary library for use in methods of the invention includes trimer pseudopeptides (or peptoids). Generally, such libraries are composed of a large number of compounds (e.g., over 10,000 compounds) distributed in pools of individual peptoids and the peptoids exhibit proteolytic stability. Trimer pseudopeptide libraries have been used in the identification and development of lead compounds, such as G-protein coupled receptor antagonists (see, for example, Blaker et al. (2000) Mol. Pharmacol. 58:399-406; Gao et al. (1999) Curr. Med. Chem. 6:375-388).

The compounds identified through screening in one or more assays, as described herein, can serve as conventional “lead compounds” or can themselves be used as potential or actual therapeutics.

In the methods of the subject invention, each compound composition is brought into contact with the biological specimen population in a manner such that the active agent of the compound composition is capable of exerting activity on at least a substantial portion of, if not all of, the individual biological specimens of the population. By substantial portion, it is meant that at least 75%, usually at least 80%, and in many embodiments as high as 90 or 95% or higher will be affected. Generally, the members of the population are in contact with each compound test agent in a manner such that the active agent of the composition is internalized by the animals. In some cases, internalization will be by ingestion, i.e. orally, such that that each compound composition will generally be in contact with the plurality of specimens by incorporating the compound composition in a nutrient medium, e.g. water, yeast paste, aqueous solution of additional nutrient agents, etc., for the biological specimens. For example, the candidate agent is generally orally administered to a fly by mixing the agent into the fly nutrient medium, such as a yeast paste, and placing the medium in the presence of the fly (either the larva or adult fly) such that the fly feeds on the medium. In some cases, members of a population are in contact with a compound by exposing the population to the compound in the atmosphere, including vaporization or aerosol delivery of the compound, or spraying a liquid containing the compound onto the animals. In some cases, members of the population (e.g., larval animals) are injected with the compound.

The compound composition may be in contact with the population of animals at any convenient stages during the life cycle of the animal. Thus, depending on the particular biological specimens, employed, the compound composition is contacted with the specimens during an immature life cycle stage, e.g. prelarval stage or larval stage, or alternatively during an adult stage, or at multiple times. Biological specimen contact with the composition may occur once or many times and administration of the compound may in an acute or a chronic mode.

In some instances, a plurality of assay mixtures are run in parallel with different agent concentrations to obtain a differential response to the various concentrations of test agent. Typically, one of these concentrations serves as a negative control, i.e., no test agent.

The invention further provides for (i) the use of agents identified by the above-described screening assays for treatment of disease in mammal, e.g., humans, (ii) pharmaceutical compositions comprising an agent identified by the above-described screening assay and (iii) methods for treating a mammal, e.g., human, with a disease by administering an agent identified by the above-described screening assays. In one embodiment, the invention provides a method of preparing a medicament for use in treatment of a disease in mammals by (a) providing a population of biological specimens (e.g., flies) with characteristics of a mammalian disease (b) using a method described herein to identify an agent expected to ameriorate the disease phenotype (e.g., an agent with an agent phenoprofile that is similar to a phenoprofile of a population of flies with a healthy phenotype) and (c) formulating the agent for administration to a mammal. In some cases, the phenotype of the population of specimens in step (a) may be characteristic of a mammalian neurodegenerative disease. The population of specimens in step (a) may be transgenic specimens and, in some cases, the expression of the transgene may result in neurodegeneration or a phenotype of a neurodegenerative disease. Genes and transgenes associated with mammalian neurodegenerative diseases and biological specimens containing such transgenes are described herein.

In one aspect, a method of preparing a medicament for use in treating a disease is provided, comprising formulating the agent for administration to a mammal, e.g., primate. For example, suitable formulations may be sterile and/or substantially isotonic and/or in full compliance with all Good Manufacturing Practice (GMP) regulations of the U.S. Food and Drug Administration and/or in a unit dosage form. See, Remington's Pharmaceutical Sciences (17th ed.) Mack Publishing Co., Easton, Pa.; Avis et al (eds.) (1993).

### EXAMPLES

### Example 1

### High Throughput Screening of Compounds Using a Fly Neurodegeneration Model

A library of compounds is screened for activity in an animal model system for neurodegeneration. The test animals are transgenic Drosophila melanogaster which express a human polypeptide associated with SCA1, ataxin-1, in all neurons. These animals, designated SCA1^{82Q}, are generated using the GAL4/UAS system to express the transgene which encodes full-length ataxin-1 82Q, an isoform of ataxin-1 with an expanded glutamine repeat (Fernandez-Funez et al. (2000)). SCA1^{82Q} flies demonstrate impaired motor performance in which they appear to lose balance, e.g., fall on their backs and have difficulty righting themselves. This impaired motor function is adult in onset and progresses over time.

In the screening assay, a population of animals, about 10-20 flies, are in optically transparent vials. Test compounds are administered to test populations by adding the test compound to a yeast paste and the yeast paste is added to the vial. The library of test compounds consists of compounds based on 2,5-diketopiperazine (DKP), is biased toward particular amines and has molecular weights generally ranging from 250-400 g/mol, as described in Szardenings et al. (1998) J. Med. Chem. 41:2194-2200. Test compounds are administered at three concentrations (approximately 0.1, 1.0 and 10 micrograms per vial) for 12 days of treatment. Two reference populations of animals in the assay are SCA182Q flies receiving no test compound (“negative reference phenoprofile”) and wild-type flies (“positive reference phenoprofile”).

Using the automated motion tracking apparatus described herein, movement of the files in the test populations and the reference populations are imaged and analyzed. In the assay, after the flies are gently tapped to the bottom, the motor activity of the flies in each population is captured in 20-50 consecutive frames using a CCD-video camera. In analysis of each frame, algorithms identify each fly as an oval, define its center and record the polar vector of the oval. Trajectories of the flies in a population are then analyzed on the basis of defined parameters, including variables such as, average speed, vertical-only speed, vertical distance, frequency of turning, trajectory count, average object size, and the variance about the mean trajectory (which identifies “stumbling” behavior). Results of these parameters are stored and assays of the populations are performed multiple times over the course of the adult life span of the flies.

Multivariate analysis is used to compare parameter results from the test populations of animals and from the reference populations and the analysis is used to define a phenoprofile associated with an test compound, i.e., agent phenoprofile and to define the reference phenoprofiles. A comparison of the agent phenoprofile to the reference phenoprofile is used to identify test compounds with activity in the test animals. Agents producing agent phenoprofiles similar to the positive reference phenoprofile and/or dissimilar to the negative reference profile are candidates for treatment of spinocerebellar ataxia in mammals.

Score Definitions for Examples 2-4.

The examples below were performed using the following score definitions.

Each movie is first scored individually to give one value per score and movie. A single movie is therefore considered to be the experimental base unit. Thereafter average values and standard errors for all scores are calculated from the movie score values for all repeats for a vial. Those averages and standard errors are the values shown in the PhenoScreen program. The data that is used in the scoring process are the trajectories of the corresponding movie. Each trajectory consists of a list of x- and y-coordinates of the position of the fly (and also size), with one list entry for every frame from when it starts moving in one frame until it stops in another.

Score definitions are as follows. The data corresponding to each score is a measure of “movement trait data”:

X-Pos: The X-Pos score is calculated by concatenating the lists of x-positions for all trajectories and then computing the average of all values in the concatenated list.

X-Speed: The X-Speed score is calculated by first computing the lengths of the x-components of the speed vectors by taking the absolute difference in x-positions for subsequent frames. The resulting lists of x-speeds for all trajectories are then concatenated and the average x-speed for the concatenated list is computed.

Speed: The Speed score is calculated in the same way as the X-Speed score, but instead of only using the length of the x-component of the speed vector, the length of the whole vector is used. That is, [length]=square root of ([x-length]²+[y-length]²).

Turning: The Turning score is calculated in the same way as the Speed score, but instead of using the length of the speed vector, the absolute angle between the current speed vector and the previous one is used, giving a value between 0 and 90 degrees.

Stumbling: The Stumbling score is calculated in the same way as the Speed score, but instead of using the length of the speed vector, the absolute angle between the current speed vector and the direction of body orientation is used, giving a value between 0 and 90 degrees.

Size: The Size score is calculated in the same way as the Speed score, but instead of using the length of the speed vector, the size of the detected fly is used.

T-Count: The T-Count score is the number of trajectories detected in the movie.

P-Count: The P-Count score is the total number of points in the movie (i.e., the number of points in each trajectory, summed over all trajectories in the movie).

T-Length: The T-Length score is the sum of the lengths of all speed vectors in the movie, giving the total length all flies in the movie have walked.

Cross150: The Cross150 score is the number of trajectories that either crossed the line at x=150 in the negative x-direction (from bottom to top of the vial) during the movie, or that were already above that line at the start of the movie. The latter criteria was included to compensate for the fact that flies sometimes don't fall to the bottom of the tube. In other words this score measures the number of detected flies that either managed to hold on to the tube or that managed to climb above the x=150 line within the length of the movie.

Cross250: The Cross250 score is equivalent to the Cross150 score, but uses a line at x=250 instead.

F-Count: The F-Count score counts the number of detected flies in each individual frame, and then takes the maximum of these values over all frames. It thereby measures the maximum number of flies that were simultaneously visible in any single frame during the movie.

### Example 2

### Motion Tracking with Wild-Type Flies

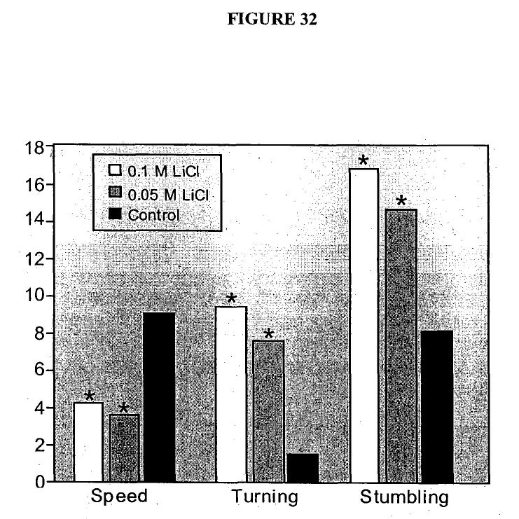

Several sets of wild-type flies were assayed under various conditions to test the motion tracking software. Lithium Chloride (LiCl), a treatment for bipolar affective disorder in humans, is also known to induce behavioral changes in Drosophila (Xia et al., 1997). In this assay, flies fed 0.1M or 0.05M LiCl exhibited a significant reduction in speed and an increase incidence of turning and stumbling compared to controls. The results of this assay are shown in the bar graph of FIG. 32.

### Example 3

### Motion Tracking with Drosophila Model of Huntington Disease

Drosophila expressing a mutant form of human Huntington (HD) have a functional deficit that is quantifiable, reproducible, and is suitable for automated high-throughput screening. Drosophila (or specimen) movements can be analyzed for various characteristics and/or traits. For example, statistics on the movements of the specimens, such as the x and y travel distance, path length, speed, turning, and stumbling, can be calculated. These statistics can be averaged for a population and plotted.

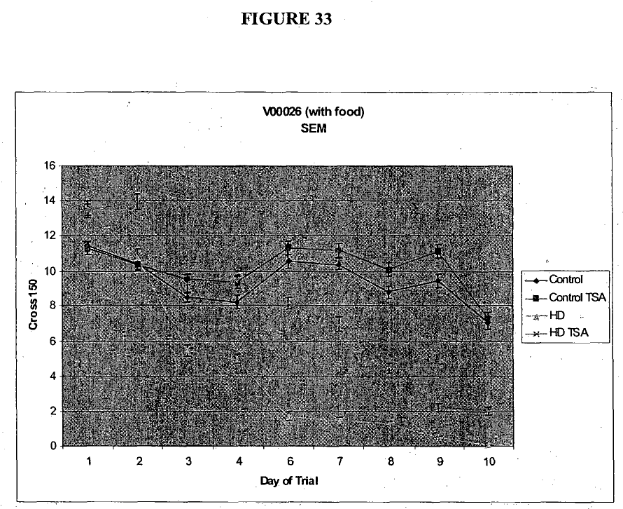

Differences between the HD model +/−drug (HDAC inhibitor, TSA) and wild type (control) +/−drug (TSA) can clearly be detected using the Phenoscreen software. Progressive motor dysfunction and therapeutic treatment with drug can be measured by various scoring parameters. Such results are shown in FIG. 33. In FIG. 33, motor performance, assessed by the Cross150 score, is plotted on the y-axis against time α-axis). The Cross150 score, or x travel distance, is equal to the number of trajectories (specimens) that cross a position at x=150 in the negative x-direction (from bottom to top of the vial) during the movie. In other words, this score measures the number of detected flies that climb above the x=150 line within the length of the movie. This graph demonstrates the potential therapeutic effect of drug (TSA) on the HD model. Error bars are +/−SEM). Control genotype is yw/elavGAL4. HD genotype is HD/elavGAL4.

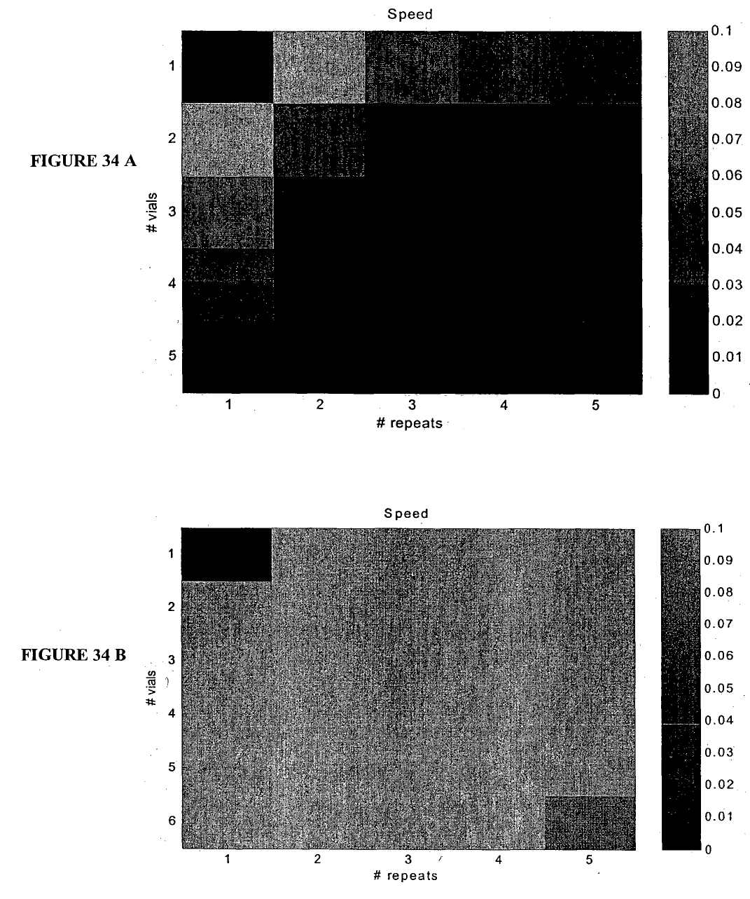

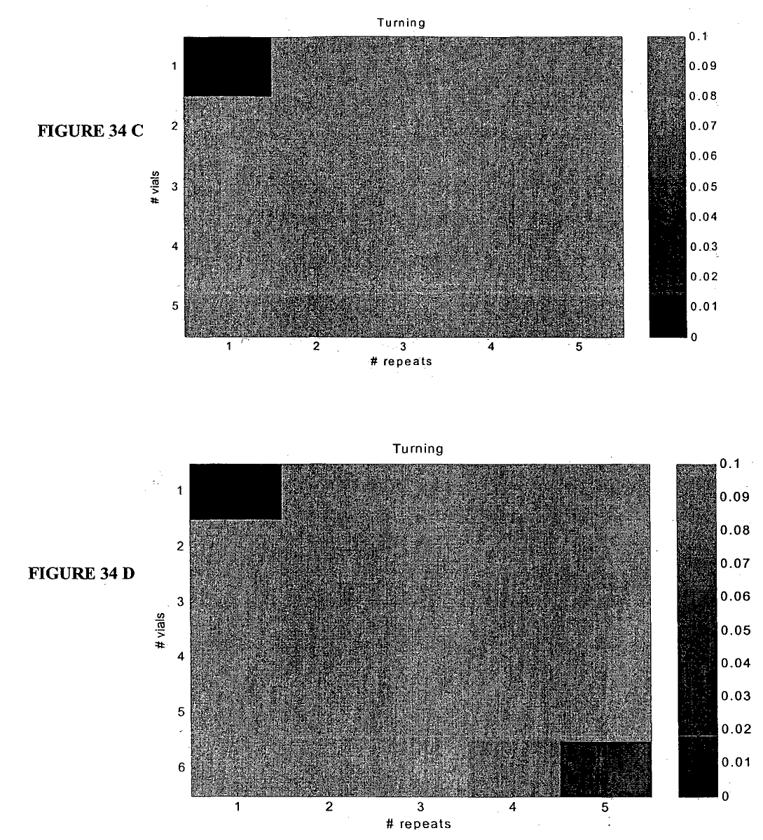

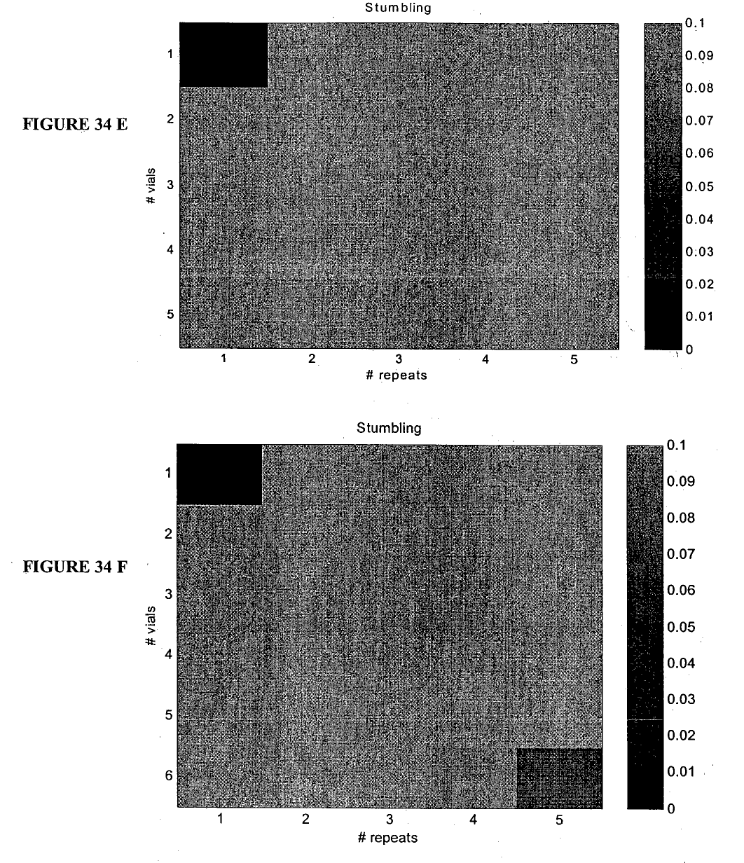

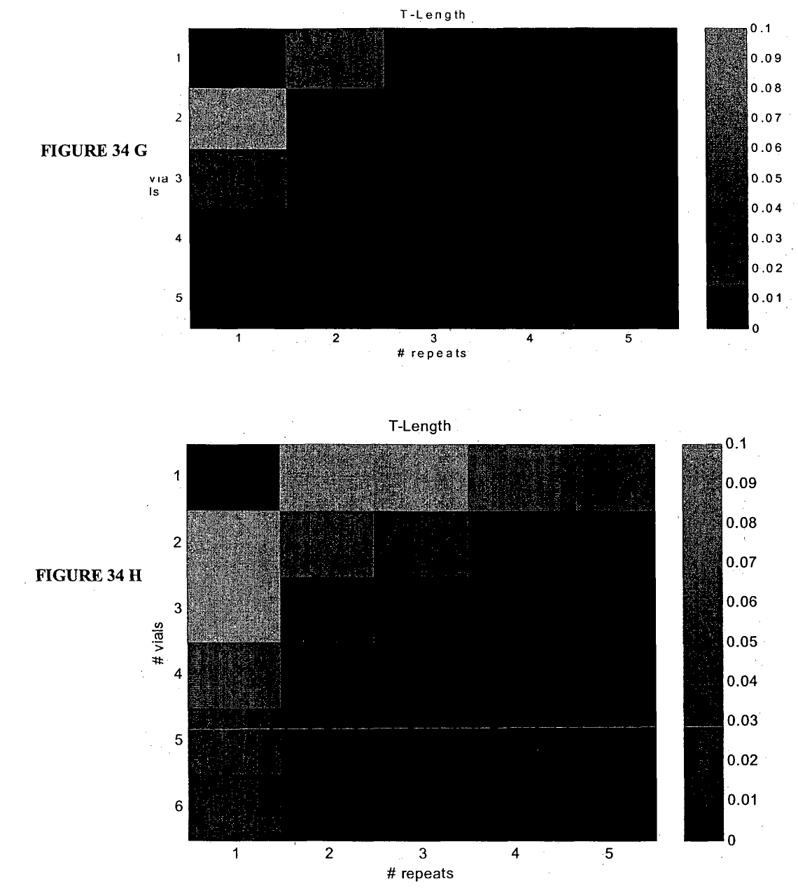

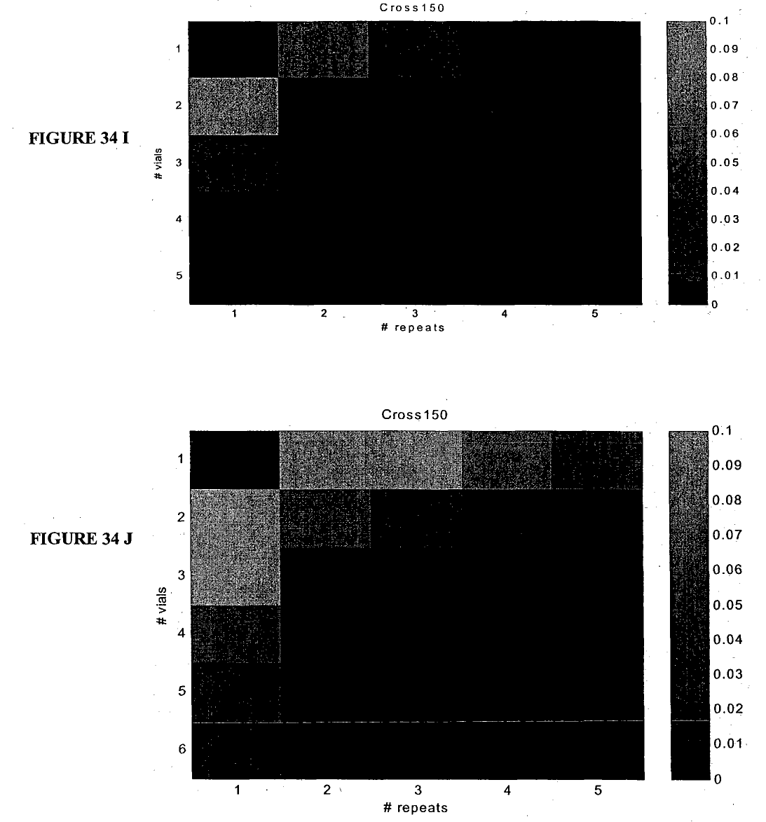

Movement characteristics of different models, or the effects of certain drugs on those models, will be distinct. FIGS. 34A-34J demonstrate (1) how well various scores define the differences between disease model and wild-type control, (2) how well the various scores detect improvements +/−drug treatment, and (3) how many replica vials and repeat videos are needed for statistically significant results. In FIGS. 34A-34J, the average p-values for each combination of a certain number of video repeats and replica vials for Test and Reference populations are shown. Lower-values are indicated by darker coloring. The lower the p-value, the more likely the score represents a significant difference between Test and Reference populations. In FIGS. 34A, 34C, 34E, 34G and 341, the Reference population is wild-type control and the Test population is the HD model. In FIGS. 34B, 34D, 34F, 34H and 34J, the Reference population is HD model without drug and the Test population is the HD model with drug (TSA). Speed is shown in FIGS. 34A and 34B, turning is shown in FIGS. 34C and 34D, stumbling is shown in FIGS. 34E and 34F, T-length is shown in FIGS. 34G and 34H, and Cross 150 is shown in FIGS. 34I and 34J.

In FIGS. 34A, 34G and 34I, Speed, T-Length, and Cross150 scores are very useful for identifying HD flies from wild-type control flies—the p-value goes down when either number of replica vials or number of repeat videos are increased, which is to be expected. Turning and Stumbling scores do not appear do give significant values not even for large number of replica vials or videos repeats. In FIGS. 34B, 34D and 34F, the scores for Speed, Turning, and Stumbling do not yield significant values. The scores that best highlight the therapeutic effect of the drug in the HD model are T-Length (FIGS. 34G and 34H) and Cross150 (FIGS. 34I and 34J). Note the striking differences between the Speed plots (FIGS. 34A and 34B). Speed is a useful score for telling apart HD flies from wild type flies, however it does not appear to be effective for telling apart HD untreated flies from HD with drug flies. Although the drug seems to restore climbing ability for HD flies to almost the same level as for wt flies, the same is not true for speed.

### Example 4

### Motion Tracking with Drosophila Model of Spinocerebellar Ataxia Type 1

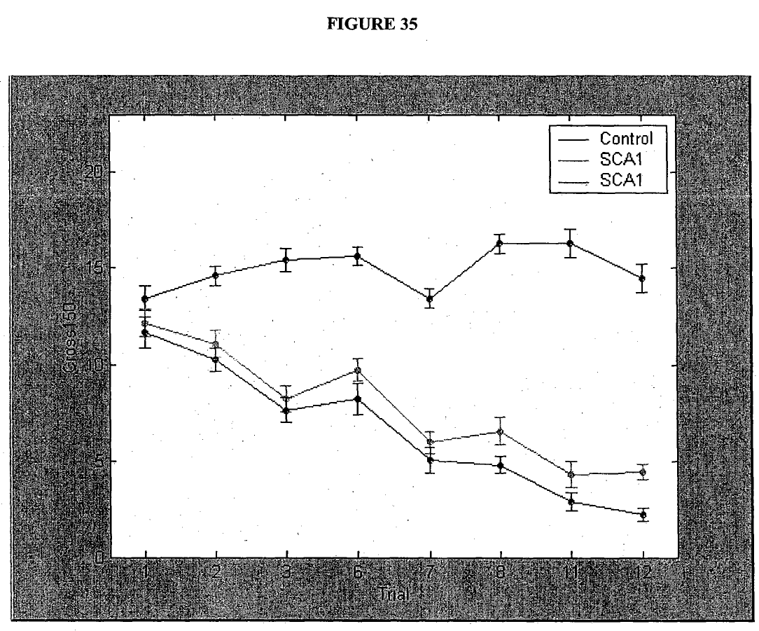

FIG. 35 shows the loss of motor performance in the SCA1 Drosophila model. SCA1 model and control trials were analyzed and plotted by Phenoscreen software. Motor performance on the y-axis (Cross150) is plotted against time on the x-axis (Trials). SCA1 model is indistinguishable from controls on first day of adult life then they decline progressively in climbing ability. The error bars are +/−SEM. Control fly genotype is yw/nirvanaGAL4. SCA1 fly genotype is SCA1/nirvanaGAL4.

What is claimed is:

1. A computer system to assess a condition of a biological specimen by studying physical traits of a sample comprising a number of biological specimens, the system comprising:
a user interface comprising a computer screen, an input interface, and a user interface portion of a processing mechanism;
the user interface comprising a specimen information input mechanism, the specimen information input mechanism comprising an association input to associate certain samples with a test population of biological specimens and other samples with a reference population of biological specimens;
an imaging system to identify changes in visible features of the specimens of the sample, the changes in visible features comprising physical trait data indicative of physical traits of the specimens; and
data storage comprising sample data and the physical trait data corresponding to the sample.

2. The computer system according to claim 1, wherein the physical trait data comprises movement trait data regarding individual biological specimens.

3. The computer system according to claim 2, wherein the movement trait data is selected from the group consisting of X-pos, X-speed, speed, turning, stumbling, size, T-count, P-count, T-length, Cross150, Cross250, and F-count.

4. The computer system according to claim 2, further comprising an animation mechanism to create a graphically represented animation of positions and movements of the individual biological specimens.

5. The computer system according to claim 1, wherein the specimen information input further comprises a grouping input to associate certain samples with each other.

6. The computer system according to claim 1, wherein the biological specimen comprises an animal.

7. The computer system according to claim 1, wherein the biological specimen comprises a fly.

8. The computer system according to claim 1, wherein the biological specimen comprises drosophila.

9. The computer system according to claim 1, wherein the condition comprises a human central nervous system condition and wherein the biological specimens comprise live transgenic non-human animals.

10. The computer system according to claim 1, wherein the specimen information input mechanism comprises a sample identification mechanism to automatically identify each sample by assigning an identifier associated with a location of placement of the sample.

11. The computer system according to claim 1, wherein the specimen information input mechanism comprises a sample identification mechanism to identify each sample with a bar code.

12. The computer system according to claim 1, wherein the specimen information input mechanism comprises a sample identification mechanism to allow a user to input and identification of each sample through computer screen input.

13. The computer system according to claim 1, wherein the specimen information input mechanism comprises an animal type input to allow a user to specify through computer screen input the type of biological specimen to be studied.

14. The computer system according to claim 1, wherein the user interface further comprises a physical trait input mechanism to allow a user to specify through computer screen input a set of physical traits of the sample to be analyzed.

15. The computer system according to claim 1, wherein the imaging system comprises a motion tracking system to track motion of the biological specimens within the sample and produce motion information, and to produce, from the motion information, behavioral trait data concerning the sample.

16. The computer system according to claim 1, wherein said data storage further comprises physical trait data corresponding to each biological specimen.

17. The computer system according to claim 1, wherein said data storage comprises sample data.

18. The computer system according to claim 1, wherein the type of specimen comprises information concerning a transgenic alteration of the biological specimen.

19. The computer system according to claim 1, wherein the specimen information input mechanism comprises a number input mechanism to input a number of samples to be assessed by the computer system, the number input mechanism being configured to receive computer screen input by a user of numbers at least as high as one hundred (100).

20. The computer system according to claim 1, wherein the specimen input mechanism comprises a imaging-based counter to count a number of specimens in the sample using an image of the sample.

21. The computer system according to claim 1, further comprising:
a first input mechanism to identify through a computer screen input a test biological specimen population;
a second input mechanism to identify through a computer screen input a reference biological specimen population;
a trait determining mechanism to cause the motion tracking system to produce test physical trait data concerning the test biological specimen population and to produce reference physical trait data concerning the reference biological specimen population;
a comparison mechanism to compare a chosen portion of the test physical trait data to a corresponding chosen portion of the reference physical trait data to produce a comparison result.

22. The computer system according to claim 20, wherein the chosen portion of the test physical trait data comprises all the test physical trait data and wherein the corresponding chosen portion of the reference physical trait data comprises all the reference physical trait data.

23. The computer system according to claim 20, wherein the chosen portion of the test physical trait data comprises substantially less than all the test physical trait data and wherein the corresponding chosen portion of the reference physical trait data comprises substantially less than all the reference physical trait data.

24. The computer system according to claim 22, wherein the system further comprises an analyzing mechanism to analyze the comparison result to assess an extent to which the chosen portion of the test physical trait data and the corresponding chosen portion of the reference physical trait data correlate to information about a human central nervous system condition.

25. The computer system according to claim 22, wherein the system further comprises an analyzing mechanism to analyze the comparison result to make an assessment regarding a treatment made to the test population and not made to the reference population.

26. The computer system according to claim 22, wherein the system further comprises an analyzing mechanism to analyze the comparison result to make an assessment regarding whether the test population has a human central nervous system disease known not to exist in the reference population.

27. The computer system according to claim 6, wherein the physical trait data comprises behavior traits of the animals.

28. The computer system according to claim 20, further comprising an analyzing mechanism to analyze the comparison result to make an assessment regarding a treatment made to the test population and not made to the reference population, the analysis of the comparison result comprising determining when a difference between data from the trait data of the test population and data from the trait data of the reference population is over a specified threshold, the analyzing mechanism comprising a decision mechanism to decide that the treatment is effective in mitigating or preventing a central nervous system condition.

29. The computer system according to claim 1, further comprising:
the user interface comprising a condition type input mechanism to allow a user to specify, through computer screen input, a specific central nervous system condition to be analyzed;
the user interface further comprising an image collection input mechanism to allow a user to specify, through computer screen input, how image data is collected and a duration over which the image data is to be collected;
the user interface further comprising a sample configuration input mechanism to allow a user to specify, through computer screen input, a number of specimens to be assessed; and
the user interface further comprising an specimen population identification input mechanism to allow a user to enter, through a computer screen input, an identification number for each specimen population comprising specimens to be tracked.

30. The computer computer system according to claim 1, where the specimen information input mechanism comprises a mechanism to present to the user on a computer screen a list of possible animal populations from which the user can choose a specimen population using the input interface.

31. The computer system according to claim 29, wherein the input interface comprises a keyboard and a cursor control device.

32. The computer system according to claim 30, wherein the cursor control device comprises a mouse.

33. The computer system according to claim 28, wherein the condition type input mechanism allows the user to choose a specific central nervous system disease and enter a set of physical traits relating to the disease.

34. The computer system according to claim 28, wherein the condition type input mechanism allows the user to enter a set of physical traits without specifying a central nervous system condition.

35. The computer system according to claim 28, wherein the imaging system comprises a calculating mechanism to calculate physical traits including path length.

36. The computer system according to claim 28, wherein the imaging system comprises a calculating mechanism to calculate physical traits including velocity.

37. The computer system according to claim 28, wherein the imaging system comprises a calculating mechanism to calculate physical traits including turning.

38. The computer system according to claim 28, wherein the imaging system comprises a calculating mechanism to calculate physical traits including stumbling.

39. A computer system to assess a human central nervous system condition by studying physical traits of a sample comprising a number of biological specimens, the system comprising:
a user interface comprising a computer screen, an input interface, and a user interface portion of a processing mechanism;
the user interface comprising an specimen information input mechanism, comprising an specimen information input to allow a user to specify through computer screen input information about the specimen to be studied;
a motion tracking system to track motion of the specimens within the sample and produce motion information, and to produce, from the motion information, physical trait data concerning a set of physical traits;
the user interface further comprising a physical trait subset input mechanism to allow a user to specify through computer screen input a subset of physical traits of the sample to be used in assessing a human central nervous system, the subset of physical traits being a subset of the set of physical traits; and
data storage comprising sample data and the produced physical trait data corresponding to the sample data.

40. A computer interface for a system to assess a human central nervous system condition by studying physical traits of a sample comprising a number of biological specimens, the computer interface comprising:
a user interface comprising a computer screen, an input interface, and a user interface portion of a processing mechanism;
the user interface comprising an specimen information input mechanism, comprising an specimen information input to allow a user to specify through computer screen input information about the specimen to be studied; and
the user interface further comprising a physical trait subset input mechanism to allow a user to specify through computer screen input a subset of physical traits of the sample to be used in assessing a human central nervous system, the subset of physical traits being a subset of the set of physical traits.

41. A machine-readable media encoded with information, the information when read by a machine causing a machine to:
receive from a user, through computer screen input, information regarding a specimen to be studied, the specimen being one of a sample of non-human biological specimens for assessment of a human central nervous system condition by studying physical traits of the specimen;
cause a motion tracking system to track motion of the specimens within the sample and produce motion information, and to produce, from the motion information, physical trait data concerning a set of physical traits; and
receive from a user, through computer screen input, a subset of physical traits of the sample to be used in assessing a human central nervous system, the subset of physical traits being a subset of a set of physical traits determined by the motion tracking system and stored in a data storage.
